SEQUENCE LISTING (20 sequences)

SEQ ID NO: 1 - DNA, 40 bp, artificial sequence
  probe for PCR reaction
aggaggaataaatgtgatctagatcacattttagaggagg

SEQ ID NO: 2 - DNA, 40 bp, artificial sequence
  probe for PCR reaction
cctcctctaaaatgtgatctagatcacatttattcctcct

SEQ ID NO: 3 - DNA, 40 bp, artificial sequence
  probe for PCR reaction
aggaggaataaatctgatctagatcagattttagaggagg

SEQ ID NO: 4 - DNA, 40 bp, artificial sequence
  probe for PCR reaction
cctcctctaaaatctgatctagatcagatttattcctcct

SEQ ID NO: 5 - DNA, 40 bp, artificial sequence
  probe for PCR reaction
aggaggaataaatgtcatctagatgacattttagaggagg

SEQ ID NO: 6 - DNA, 40 bp, artificial sequence
  probe for PCR reaction
cctcctctaaaatgtcatctagatgacatttattcctcct

SEQ ID NO: 7 - DNA, 40 bp, artificial sequence
  probe for PCR reaction
aggaggaataaatctcatctagatgagattttagaggagg

SEQ ID NO: 8 - DNA, 40 bp, artificial sequence
  probe for PCR reaction
cctcctctaaaatctcatctagatgagatttattcctcct

SEQ ID NO: 9 - DNA, 28 bp, artificial sequence
  probe for PCR reaction
aataaatgtgatctagatcacattttag

SEQ ID NO: 10 - DNA, 28 bp, artificial sequence
  probe for PCR reaction
ctaaaatgtgatctagatcacatttatt

SEQ ID NO: 11 - DNA, 28 bp, artificial sequence
  probe for PCR reaction
aataaatctgatctagatcagattttag

SEQ ID NO: 12 - DNA, 28 bp, artificial sequence
  probe for PCR reaction
ctaaaatctgatctagatcagatttatt

SEQ ID NO: 13 - DNA, 28 bp, artificial sequence
  probe for PCR reaction
aataaatgtcatctagatgacattttag

SEQ ID NO: 14 - DNA, 28 bp, artificial sequence
  probe for PCR reaction
ctaaaatgtcatctagatgacatttatt

SEQ ID NO: 15 - DNA, 28 bp, artificial sequence
  probe for PCR reaction
aataaatctcatctagatgagattttag

SEQ ID NO: 16 - DNA, 28 bp, artificial sequence
  probe for PCR reaction
ctaaaatctcatctagatgagatttatt

SEQ ID NO: 17 - DNA, 21 bp, artificial sequence
  probe for PCR reaction
gaccatgattacgccaagcta

SEQ ID NO: 18 - DNA, 20 bp, artificial sequence
  probe for PCR reaction
cagctttcatccccgatatg

SEQ ID NO: 19 - DNA, 19 bp, T7 bacteriophage
ctaatacgactcactatag

SEQ ID NO: 20 - DNA, 28 bp, E. coli
aataaatgtgatctagatcacattttag

(19) United States
(12) Patent Application Publication
(10) Pub. No.: US 2013/0337579 A1
(43) Pub. Date: Dec. 19, 2013

(54) Methods for Determining Protein Ligand Binding

(71) Applicants: Vincent T. Lee, Silver Spring, MD (US); Kevin G. Roelofs, Berwyn Heights, MD (US); Gregory Donaldson, Silver Spring, MD (US)

(72) Inventors: Vincent T. Lee, Silver Spring, MD (US); Kevin G. Roelofs, Berwyn Heights, MD (US); Gregory Donaldson, Silver Spring, MD (US)

(73) Assignee: UNIVERSITY OF MARYLAND, COLLEGE PARK, College Park, MD (US)

(21) Appl. No.: 13/819,010

(22) PCT Filed: Sep. 6, 2011
(86) PCT No.: PCT/US11/50522

§ 371 Date: Aug. 30, 2013

Related U.S. Application Data
(60) Provisional application No. 61/380,005, filed on Sep. 3, 2010.
(60) Provisional application No. 61/470,782, filed on Apr. 1, 2011.

(51) Int. Cl. G01N 33/566

(52) U.S. Cl. CPC G01N 33/566
USPC 436/501

(57) ABSTRACT

Provided is a high-throughput differential radial capillary action of ligand assay (DRaCALA) that can be used to detect ligand binding to a protein. The assay is rapid, quantitative and allows detection of protein-ligand interactions for both purified proteins and proteins expressed in whole cells, which eliminates the need for protein purification. The method does not require a wash step, and can be performed without a drying step and without the aid of electrophoretic techniques. The method entails separating unbound ligand from bound ligand by placing a liquid composition that contains or is suspected of containing a protein and a detectably labeled ligand on a dry porous membrane to obtain a location on the membrane that contains the protein. Ligand that is bound to the protein does not migrate away from the location while unbound ligand radially migrates away from the location by capillary action, which separates unbound from bound ligand. The method includes determining whether a ligand binds to one or more proteins and whether a test composition contains a protein.

### CROSS-REFERENCE TO RELATED APPLICATIONS

This application claims priority to U.S. provisional application Ser. No. 61/380,005, filed Sep. 3, 2010, and U.S. provisional application Ser. No. 61/470,782, filed Apr. 1, 2011, the entire disclosures of each of which are incorporated herein by reference.

### FIELD OF THE INVENTION

This invention relates generally to the field of ligand protein binding and more particularly to a method for determining ligand protein binding that uses capillary action for separation of bound and unbound ligand.

### DESCRIPTION OF RELATED ART

Interactions of various ligands with proteins, such as with protein receptors, are critical in biological signaling both between cells and within individual cells. Examples of intercellular signaling mediated by small molecules include quorum signaling in bacteria, hormone and neurotransmitter responses in endocrine systems of animals, and auxin and abscisic acid regulation in plants. Intracellular signaling also involves regulatory protein binding molecules such as calcium and cyclic nucleotides (e.g. cAMP, cGMP, and cyclic-di-GMP (cdiGMP)) In fact, nucleotide receptors are often targets for therapeutic intervention. Thus, these protein-ligand interactions have important implications in modern drug design and use. Considering that many protein-ligand interaction pairs represent potential targets of pharmaceutical intervention in disease or agriculture, there is an urgent need to collect qualitative and quantitative data for such protein-ligand interactions in a high throughput manner. Current efforts in metabolomics are directed at cataloging the presence of various metabolites through mass spectrometric analysis of biological samples. However, this approach lacks the ability to confirm interactions with protein partners and therefore fails to reveal functional significance. Thus, the study of the interactions of a specific metabolite with all available cellular proteins, which we term “metabolite interactomics”, has been limited by the available assay systems. Current assays for specific protein-ligand interactions, including equilibrium dialysis, filter binding assays, ultracentrifugation, isothermal calorimetry (ITC), surface plasmon resonance, and many other assays are not high-throughput as they are limited by sample processing time, equipment requirements, and assay-specific manipulations. Protein array technology requires purified proteins fixed on solid support. Although protein array technology is feasible and quite powerful, protein purification in large scale is limited by individual protein characteristics that often hinder isolation of functionally active proteins. Further, protein array technology is limited to only a few laboratories capable of performing mass parallel purification of functional proteins and arraying them. Thus, there is an ongoing and unmet need for improved methods of determining interactions between ligands and proteins. The present invention meets these and other needs.

### SUMMARY OF THE INVENTION

We have developed a technique referred to herein as high-throughput differential radial capillary action of ligand assay (DRaCALA) that can be used to detect binding and to quantitate the fraction of a small molecule ligand that is bound to a protein of interest. DRaCALA is rapid, quantitative and allows detection of protein-ligand interactions for both purified proteins and proteins expressed in whole cells, thus bypassing the requirement for protein purification. Unlike other protein-ligand detection systems, DRaCALA does not require a wash step, so the total ligand available to protein is quantifiable resulting in an accurate, simple and precise measure of the fraction of ligand bound. The method can be performed without a drying step, and without the aid of electrophoretic techniques. It is a very rapid assay and is thus readily adaptable to high-throughput techniques. Ligands of widely varying sizes can be analyzed using the method.

In general, the invention comprises a method of separating unbound ligand from bound ligand. The method comprises the general steps of placing a liquid composition comprising a protein and a detectably labeled ligand on a dry porous membrane to obtain a location on the membrane comprising the protein. Ligand that is bound to the protein does not migrate away from the location while unbound ligand radially migrates away from the location, thereby effecting separation of the unbound detectably labeled ligand from the bound detectably labeled ligand. Ligand binding to the protein results in detectable label in an inner area of a pattern on the membrane. The inner area has greater signal intensity from the detectable label than the signal intensity from the remainder of the total area of the pattern. Conversely, if the detectable label does not specifically bind to the protein, the detectable label is present in a pattern which lacks the inner area having greater signal intensity than the signal intensity from the total area of the pattern.

In certain non-limiting embodiments, the method provides a method for determining whether a ligand binds to a protein. This embodiment comprises placing a liquid test composition comprising the protein and a detectably labeled ligand on a dry porous membrane and allowing capillary action based radial migration of unbound detectably labeled ligand on the membrane. Based on the localization of the detectably labeled ligand on the membrane, whether or not the detectably labeled ligand binds to the protein is determined. If the detectable label binds to the protein, the detectable label is present in an inner area of a pattern on the membrane which has greater signal intensity than the signal intensity from the remainder of the total area of the pattern. If the detectable label does not bind to the protein, the detectable label is present in a pattern which lacks an inner area having a greater signal intensity than the signal intensity from the total area of the pattern.

Also provided is a method for determining whether a test composition comprises a protein. This embodiment comprises placing on a dry porous membranes liquid test composition which may or may not comprise the protein, but does comprise a detectably labeled ligand which specific affinity for the protein. Allowing radial migration of unbound detectably labeled ligand on the membrane results in a pattern that has an inner area of greater signal intensity that the rest of the area of the pattern if the protein is present and lacks such an inner area of greater signal intensity if the protein is absent.

Also provided is a method for determining whether a ligand binds to any of a plurality of proteins. This embodiment comprises placing a series of liquid test compositions each comprising a distinct protein and a detectably labeled ligand on separate locations of a dry porous membrane. Allowing radial migration of unbound detectably labeled ligand at the separate locations on the membrane results in a pattern at each location that indicates the presence or absence of the protein. Again, each pattern will have an inner area of greater signal intensity than the rest of the area of the pattern if the protein is present and will lack such an inner area of greater signal intensity if the protein is not present.

The skilled artisan will recognize that the method is adaptable for a variety of assays that can function to compare the affinity of any particular ligand with one or multiple other ligands for any particular protein. For example, in one embodiment, a detectably labeled ligand can be subjected to competition assays with a plurality of unlabelled ligands to identify ligands with greater (or lesser) affinity for the protein. Such assays could be repeated to identify ligands with increasingly improved (or weakened) affinity for the protein.

### BRIEF DESCRIPTION OF THE FIGURES

FIG. 1. Principle of Differential Radial Capillary Action of Ligand Assay (DRaCALA). (A) Schematic representation of DRaCALA assay upon application of protein-ligand mixture onto nitrocellulose and capillary action. Protein (P), ligand (L) and protein-ligand complexes (PL) distribution during the assay is shown. (B) Equations used to analyze DRaCALA data for fraction bound (F_{B}) for purified proteins. For an explanation of the apparent edge effect at the capillary migration front, 8 see FIG. 8.

FIG. 2. Detection of specific protein-ligand interactions by DRaCALA. (A) DRaCALA images of interactions between purified proteins (20 μM) incubated with 500 nM ¹⁴C-cAMP, 4 nM ³²P-ATP, or 4 nM ³²P-cdiGMP. Protein-ligand mixtures were spotted on nitrocellulose and allowed to dry prior to imaging using a Fuji FLA7100 phosphorimager. Cognate protein-nucleotide combinations are indicated by arrowheads. MBP was used as a negative control. (B) DRaCALA images of competition assays assessing the ability of 1 mM of the indicated cold nucleotides to compete with binding interactions between 4 nM ³²P-cdiGMP and 2.5 μM HisMBP-Alg44_{PilZ}. (C) Graph of fraction bound for each sample in FIG. 1B with averages indicated by a horizontal bar. NC indicates no competitor. P values were determined by a Student's t-test for significant differences when compared to the no-competitor (NC) control for three independent experiments. For total intensity of each DRaCALA spot in FIGS. 2A and 2B, see Tables 1 and 2, respectively.

FIG. 3. Determination of K_{d} and k_{off} by DRaCALA. (A) DRaCALA images used for K_{d} determination for the interaction of Alg44_{PilZ} and cdiGMP. His-MBP-Alg44_{PilZ} was varied from 100 μM to 6 nM and the ³²P-cdiGMP was held constant at 4 nM. Representative images of six sets of DRaCALA experiments are shown for 40 nM to 25 μm of Alg44 protein. (B) Fraction bound from data in panel A plotted as a function of [MBP-Alg44_{PilZ}] and the best-fit line was determined by nonlinear regression using the indicated equation. A no protein control was also plotted. The fitting program varied both K_{d} and Bₘₐₓ to obtain the best fit indicated by the solid line. (C) k_{off} was determined by spotting protein-ligand mixtures onto nitrocellulose at various times post-addition of 1 mM cold cdiGMP to a mixture of 4 nM ³²P-cdiGMP and HisMBP-Alg44_{PilZ}. (D) The time course of decrease of F_{B} from analysis of the data in panel C was fitted to a single exponential decay, indicating k_{off}. For total intensity of each DRaCALA spots in FIGS. 3A and 3C, please see Tables 3 and 4, respectively.

FIG. 4. Detection of specific protein-ligand interaction in whole cell lysates by DRaCALA. (A) Images of Alg44_{PilZ} interaction with 4 nM ³²P-cdiGMP and either 1 mM of cold cdiGMP(C) or GTP(G) with purified proteins or when expressed in E. coli BL21(DE3). (B) Graph of ³²P-cdiGMP binding by whole cell lysate samples (open circles) and purified proteins (closed inverted triangles) in FIG. 4A with the average indicated by a horizontal bar. P values were determined by a Student's t-test for significant differences when compared to the no-competitor (NC) control for three independent experiments. For total intensity of each DRaCALA spot in FIG. 4B, please see Table 5. (C) Graph of ³²P-cdiGMP binding by purified MBP-Alg44_{PilZ}, purified MBP-Alg44_{PilZ} added to BL21 whole cell lysates, and whole cell lysates of BL21(DE3) overexpressing MBP-Alg44_{PilZ}. Protein concentrations were determined by separation on SDS-PAGE and staining with Coomassie blue (FIG. 9).

FIG. 5. Analysis of cdiGMP binding proteins in various organisms. B_{Sp} of whole cell lysates are shown as a heat map using the range indicated in the legend. (A) Equations used to analyze DRaCALA data for specific binding (B_{Sp}) for whole cell lysates or tissue extracts. (B) Plate 1 is the analysis of cdiGMP binding by lysates from P. aeruginosa isolates. Specific strains discussed in the text are indicated by arrows. Sources of all strains in plates 1 and 2 as well as the raw data for each lysate are shown in Table 6. (C) Plate 3 is the analysis of cdiGMP binding by lysates from various organisms. Plate numbers, column numbers and row letters correspond to the strains and organisms listed in Tables 6 and 7.

FIG. 6. Demonstration of DRaCALA. (A) Ligand distribution in the absence of protein when spotted on nitrocellulose. (B) Coomassie stained MBP immobilized on nitrocellulose. Pencil marks drawn before staining to indicate darker protein spot and total capillary action. (C) DRaCALA image of E. coli BL21(DE3) whole-cell lysates overexpressing MBP or MBP-Alg44_{PilZ} incubated with 8 nM ³²P-cdiGMP and spotted onto nitrocellulose. (D) Graph of DRaCALA spots from FIG. 6C.

FIG. 7. Dot blot analysis of cdiGMP binding to Alg44_{PilZ}. MBP-Alg44_{PilZ} at the indicated concentration was mixed with 4 nM of ³²P-cdiGMP and incubated for 10 minutes. Samples were applied to the dot apparatus and washed with 10 mM Tris, pH 8.0 and 100 mM KCl. The filter was dried and exposed to phosphorimager screen. (A) Image of dot blot experiment performed in triplicate. (B) ³²P counts graphed against each concentration of Alg44_{PilZ}.

FIG. 8. Edge and annulation effects of DRaCALA. (A) Schematic for the basis of the edge effect due to evaporation. (B) Correction factor for edge effect. (C) Schematic indicating the basis of the annulation of the protein signal. The abbreviations for protein (P), ligand (L) and protein-ligand complexes (PL) are used in the diagram.

FIG. 9. Protein pattern of purified and expressed MBP-Alg44_{PilZ}. Coomassie stained PAGE of two-fold serial dilutions of MBP-Alg44_{PilZ} as (A) a purified protein in cdiGMP binding buffer, (B) a purified protein added to BL21 WCL, or (C) an overexpressed protein in BL21 cells. Protein concentration for the purified protein in (A) and (B) was determined as described in the material and methods. The protein concentrations of the MBP-Alg44_{PilZ} in whole cell lysates (C) were estimated based on comparison to (A) and (B).

FIG. 10. Binding of cdiGMP to whole cell lysates expressing soluble or insoluble cdiGMP binding proteins. (A) BL21(DE3) cells expressing the indicated proteins were analyzed by PAGE and coomassie staining of whole cell (W) and soluble (S) fractions. Arrows indicate the overexpressed protein of the correct molecular weight in whole cell lysates. Molecular weights of proteins are indicated on the left in kilodaltons. (B) Spots of BL21 cells overexpressing the indicated proteins were assayed for cdiGMP binding by DRaCALA. (C) Quantification of the data shown in (B). (D) Binding of ³²P-cdiGMP to various dilutions of lysates of BL21(DE3) cells expressing the indicated proteins.

FIG. 11. B_{Sp} distribution of P. aeruginosa strains from different sources. B_{Sp} of ³²P-cdiGMP for P. aeruginosa isolates of different origins with mean and standard deviation. The mean±S.D. is noted above each group; no significant differences were observed.

### CF—Cystic Fibrosis, UTI—Urinary Tract Infection, ATCC—American Tissue Culture Collection.

FIG. 12. Effect for protein concentration of whole cell lysates on the ability to detect specific binding. Bacterial lysates were prepared as described in the Examples below. Extracts were diluted such that the A₂₈₀ is normalized to 60. Then lysates were diluted to the indicated A₂₆₀ and tested for ³²P-cdiGMP binding by DRaCALA.

FIG. 13. Detection of protein-DNA interaction by differential radial capillary action of ligand assay (DRaCALA). (A) Phosphorimager visualization of DRaCALA spots of indicated proteins at 100 nM mixed with 4 nM ³²P-labelled ICAP fragments and 200 μM cAMP show distributions of the radioligand that are diffused and homogenous (no protein, MBP) or sequestered (CRP). (B) The fraction bound was quantified using the formula in the Methods and error bars indicate the standard deviation for three spots.

FIG. 14. CRP binding to specific DNA sequences detected by DRaCALA. (A) The sequence of the 28 bp ICAP site (SEQ ID NO:20). The positions perturbed in this study are marked in red. Names of mutant versions are listed next to the point mutations that define them. Equivalent nomenclature for mutants from Gunasekera, et al. 1992 is indicated in parentheses (Gunasekera, A., Ebright, Y. W. and Ebright, R. H. (1992) DNA sequence determinants for binding of the Escherichia coli catabolite gene activator protein. J Biol Chem, 267, 14713-14720). (B) DRaCALA spots for direct binding of 100 nM CRP to 4 nM of ICAP, 8:G-C, 10:G-C, and 8,10:G-C probes with 200 μM cAMP are shown above the graphed quantification of fraction bound. (C) Binding of the ICAP probe to CRP was subjected to competition by unlabelled probes at 10, 100, or 1000 times the concentration of the radioligand. All error bars represent standard deviation of three spots. DRaCALA spots shown above their respective conditions are separate images consolidated to fit the graph.

FIG. 15. DRaCALA can be used to determine affinity and kinetics. (A) The affinity of CRP to the ICAP binding site reconstituted from annealed oligonucleotides was determined by the ability of serially diluted CRP to sequester 4 pM ³²P-labeled ICAP probe in the presence of 0, 200 nM, or 200 μM cAMP. K_{d} values are reported in Table 9. (B) The observed off-rate, k_{off}=2.6±0.40×10⁻³ s⁻¹ (S.D.), was measured by adding 1000-fold unlabeled competitor to 5 nM CRP with 5 pM ICAP oligonucleotide probe and spotting at different time points. All error bars represent the standard deviation of three spots.

FIG. 16. Whole plasmids carrying ICAP bind CRP specifically in DRaCALA. (A) 50 pM individual plasmids with lx, 3×, or 5× wild-type binding sites or 3× mutant binding sites cloned in series were tested for binding in the presence of 100 nM CRP and 200 μM cAMP. (B) Specificity was determined by competition of binding to ³²P-labeled 1× wild-type plasmid with unlabeled PCR products. Competitors used were 1×ICAP, 3×8:G-C, 3×10:G-C, 3×8,10:G-C. All error bars represent standard deviation of three spots with a representative spot (spot images consolidated to fit graph) shown above each column.

FIG. 17. Affinity and kinetics of DNA-binding determined using 5 pM whole plasmid probe with a single ICAP site. (A) Graphs of fraction of ICAP plasmid bound by various concentrations of CRP with indicated levels of cAMP (K_{d} reported in Table 9). (B) Graph of observed off-rate of k_{off}=4.8±0.17×10⁻⁴ s⁻¹ (S.D.) for ICAP plasmid generated by adding 1000-fold unlabelled PCR product (lx ICAP) competitor to 5 nM CRP with plasmid probe and spotting at time points over three hours. All error bars represent standard deviations of three spots.

FIG. 18. Bioconjugate DNA probes. Bioconjugate probes were generated by PCR with 5′-biotinylated primers. (A) Four probes (ICAP and 8,10:G-C with and without biotin) were tested for binding to CRP, streptavidin, and MBP. A mix of 50 pM ³²P-labeled probe, 100 nM protein, and 200 μM cAMP was spotted on 0.8 micron nitrocellulose and phosphor images of the spots are shown. (B) The ICAP-biotin probe affinity for streptavidin in PBS was determined by DRaCALA with 100 pM probe (K_{d}=4.0±0.6×10⁻¹⁰ M). (C) Binding of 10 nM streptavidin to the ICAP-biotin probe was competed with serial dilutions of free biotin (IC₅₀=3.3×10⁻⁸ M).

FIG. 19. Vc2* RNA binding to ³²P-cdiGMP is detected by DRaCALA. (A) Spots visualized by phosphorimager with streptavidin used to immobilize biotinylated RNA. The binding reaction contained 4 nM ³²P-cdiGMP, 1 μM RNA, and 200 nM streptavidin in buffer (10 mM KCl, 10 mM sodium cacodylate, 3 mM MgCl₂). (B) The affinity of Vc2*-biotin RNA for cdiGMP was determined with both EMSA and DRaCALA by diluting RNA in the binding reaction. The fraction bound is normalized such that 1.0 represents maximal binding. The DRaCALA-obtained affinity was K_{d}=7.8±1.9×10⁻⁹ M and the apparent affinity in EMSA was K_{d}=9.8±1.6×10⁻⁹ M.

FIG. 20. Small detectable molecules exhibit variable mobility by capillary action through nitrocellulose. 5 μl of given concentration of each molecule was spotted: 3 nM ³²P-ATP, 10 μM TNP-ATP, 200 μM FITC-NP, 250 μM crystal violet, 300 μM Coomassie, 200 μM TRITC, 500 μM propidium iodide, 250 μM EtBr, 250 μM EtBr with 1 μM DNA.

FIG. 21. Binding of 10 nM streptavidin to 100 pM ³²P-ICAP-biotin probe measured over time after addition of 100 μM free biotin.

### DESCRIPTION OF THE INVENTION

Interactions of proteins with ligand of various kinds, such as low molecular weight ligands (i.e., metabolites, co-factors and allosteric regulators), as well as various polynucleotides, are important determinants of a variety of biological functions, including but not limited to metabolism, gene regulation and cellular homeostasis. For example, pharmaceutical agents of many types often target ligand-protein interactions to interfere with regulatory and other biological pathways.

In the present invention, we have developed a rapid, precise, and high-throughput capable method for qualitatively or quantitatively determining protein-ligand interactions. One important benefit of the invention is that no washing step is required. Additional benefits include but are not necessarily limited to the fact that the method can be performed without drying the porous substrate after contacting it with a protein and detectably labeled ligand. Thus, the lack of a drying step is but one feature that differentiates the present method from other methods for separating compounds from one another, such as thin layer chromatography. Further, the method can be performed without application of an electrical gradient (and thus is not an electrophoretic method). It is considered that the method requires no separation technique other than capillary action which can mobilize the ligand away from the protein that is attached to the substrate. Further, we demonstrate that the method can be performed without a need to purify the protein, although the use of purified protein is also a useful aspect of the invention.

This new method (DRaCALA) is based at least in part on the ability of a dry, porous substrate, such as nitrocellulose, to separate free ligand from bound protein-ligand complexes. Without intending to be constrained by any particular theory, it is considered that the porous substrate used in the method of the invention sequesters proteins and bound ligand at the site of application, whereas free ligand is mobilized by bulk movement of a solvent through capillary action. Thus, and again without intending to be restricted by theory, it is considered that by capillary action, free ligand moves outward from the initial spot while the proteins and bound ligands are immobilized by hydrophobic interactions with the nitrocellulose membrane. This allows differentiation of bound and unbound ligand based on mobility due to capillary-action. The advantages of DRaCALA over traditional filter-binding assays include but are not necessarily limited to the capability to have the total amount of ligand in samples measured, which is considered to be at least in part because there is no wash step required. Further, it is considered that the speed of DRaCALA allows kinetic measurements at near equilibrium conditions and provides for ease of varying parameters to obtain multiple data points. Further still, in various embodiments, the visual output of the method allows rapid assessment of molecular interactions. Moreover, we demonstrate that quantitative measurements of protein-ligand interaction, such as fraction bound, can be readily calculated from measurements of four parameters: the total area, the total intensity, the sequestered area, and the sequestered intensity. Thus, the simplicity of DRaCALA gives it potential for general applicability.

In one embodiment we demonstrate that DRaCALA allows detection of specific interactions between nucleotides and their cognate nucleotide binding proteins. We also show that DRaCALA allows quantitative measurement of dissociation constants (K_{d}) and dissociation rates (k_{off}). Furthermore, we show that DRaCALA can detect the expression of proteins in whole cell lysates. This demonstrates the power of the method to bypass the prerequisite for protein purification. In particular, we demonstrate the DRaCALA method by analysing cdiGMP signaling in 54 bacterial species from 37 genera and 7 eukaryotic species. These studies reveal the presence of potential specific nucleotide binding proteins in 21 species of bacteria, including four unsequenced species. The ease of obtaining metabolite-protein interaction data using the DRaCALA assay will accordingly facilitate rapid identification of protein-metabolite and protein-pharmaceutical interactions in a systematic and comprehensive approach.

In addition to low-molecular weight ligands, we applied the method of the invention to DNA-protein interactions using the well characterized interaction between E. coli cyclic AMP receptor protein (CRP) and its DNA binding site ICAP. CRP is a transcription factor that has regulatory function at approximately 200 sites on the E. coli genome. CRP binds cAMP and cGMP, but DNA binding and transcriptional activation by CRP is solely dependent on cAMP binding. A 28 bp symmetrical synthetic consensus sequence, called ICAP, binds CRP with the greatest affinity. Through filter-binding assays, the affinity of the CRP-ICAP interactions and the contributions of specific nucleotides (such as guanines at positions 8 and 10 and the cytosines at positions 19 and 21) have been previously defined. In the present invention, DRaCALA is shown to allow quantification of CRP-ICAP interactions using detectably labeled oligonucleotides. Specificity of binding and competition studies were performed, and furthermore, the method was used to obtain measurements of both affinity and kinetics. Much larger DNA probes derived from whole plasmids were tested in the same way. Thus, it is expected that DNA could function as a carrier molecule for studying interactions between a protein and a molecule covalently linked to a polynucleotide, such as DNA. This also allows easy indirect labeling of molecules that are more difficult to labeled DNA. In another embodiment, immobilization of nucleic acids with the biotin-streptavidin system is shown to allow study of small molecule interactions with RNA (riboswitches). We show here the different ways DRaCALA can be used to study molecular interactions with nucleic acids including protein-nucleic acid and riboswitch-small ligand interactions, as well as non-nucleic acid ligands. Further, there is evidence that polynucleotides could serve as a label and carrier for any molecule that can be conjugated to them. Because bioconjugate PCR allows specific immobilization of biotinylated nucleic acids, the assay can be used with nucleic acids as the immobile and/or the mobile piece in binding studies. These manipulations of the mobility of molecules provide a window to the many potential uses of this assay. Additionally, the ease of running DRaCALA (little volume needed, no wash step, inexpensive materials, and in certain aspects a visual readout) makes it amenable to usage as a portable rapid diagnostic tool in a “lab-on-paper” design.

In general, the invention comprises a method of separating unbound ligand from bound ligand by: placing a liquid composition comprising a detectably labeled ligand and a protein on a dry porous membrane. Detectably labeled ligand that binds to the protein does not migrate away from the location of the protein on the membrane, while detectably labeled ligand that does not bind to the protein radially migrates away from the location of the protein. Thus, the method effectuates separation of the unbound ligand from the bound ligand.

All aspects of the invention can be performed without a wash step.

In each aspect of the invention, the test composition comprising the protein can also comprise the detectably labeled ligand, or the protein and the ligand may be placed on the membrane sequentially, so long as the protein is placed on the membrane first.

In one aspect the invention provides a method comprising: a) placing a test composition comprising a protein and a detectably labeled ligand on a dry porous membrane; b) allowing radial migration of unbound ligand on the membrane; and c) based on the localization of the detectable label on the membrane, determining whether or not the ligand binds to the protein.

The ligand that is used in the method of the invention is not particularly limited. All that is required is that the ligand be capable of being mobilized via capillary action. In this regard, we demonstrate that the ligand can be of a low molecular weight, or it can be quite large. Low molecular weight ligands are considered to be those having a molecular weight of up to 250 daltons. Thus, in various embodiments, the ligand can have a molecular weight that is not more than from 5 to 250 daltons, inclusive, and including all digits and ranges there between. However, we demonstrate that a low molecular weight ligand is not required for the method to function, since the invention is able to discriminate between 3.5 kilobase DNA plasmids with a molecular weight of over a megadalton. Therefore, the mobility of the ligand and its size are not necessarily directly correlative. Accordingly, the ligand can be any particular ligand which binds with specificity to any particular protein.

In one embodiment, the ligand of interest is conjugated to a detectably labeled polynucleotide. Thus, in this embodiment, the detectably labeled polynucleotide alone is not considered to be the ligand. Rather, it is the ligand of interest that is considered to be the detectably labeled ligand.

In various non-limiting embodiments, the ligand can be an agent that can affect one or more biological processes via protein binding. Thus, the ligand can be an antagonist or an agonist of a receptor. The ligand can be a pharmaceutical agent, including but not limited to a psychoactive pharmaceutical agent, a chemotherapeutic agent, an agent that affects cardiovascular function, the endocrine system, the digestive system, inflammation or other immune system responses, cognitive functioning, oxidative stress, enzyme function, wound healing, or any other biological process, which include but are not necessarily limited to ligands which have antibiotic, antiviral, and/or effects on any other infectious agent, including by not necessarily limited to fungal pathogens and/or parasitic pathogens such as protozoan and helminthic pathogens. Further, the mobility of the ligand can be modulated via changes in the liquid composition in which it is present when applied to the porous substrate. In connection with this, the liquid composition comprising the ligand can be hydrophilic, such as an aqueous solution, or it can be of a hydrophobic character. The ligand can be in a solution, suspension, dispersion, emulsion, or any other state in a liquid composition that will permit the ligand to be mobilized through the porous substrate due to capillary action if it is not bound to the protein. The solvent composition could also be changed by addition of detergents, salts, and other agents may be added to alter the relative behavior of the protein and ligand on the solid support.

As will be apparent from the description and Examples presented below, the detectably labeled ligand can be a polynucleotide. The polynucleotide is not particularly limited and can be linear, circular or branched. It can be fully or partially double or single stranded. In various embodiments, the polynucleotides are endogenous to or are derived from prokaryotes, eukaryotes, or viruses. In one embodiment, the polynucleotide is a plasmid that can be replicated by bacteria. The polynucleotide can be RNA or DNA. The polynucleotide can be any RNA, including but not necessarily limited mRNA, tRNA, rRNA, a riboswitch, an apter comprising a polynucleotide, and microRNA. In addition to being detectably labeled, the polynucleotide can comprise other modifications. For instance, the polynucleotides can comprise RNA:DNA hybrids. Other modifications that can be comprised by the polynucleotides include but are not limited to modified ribonucleotides or modified deoxyribonucleotides. Such modifications can include without limitation substitutions of the 2′ position of the ribose moiety with an —O— lower alkyl group containing 1-6 saturated or unsaturated carbon atoms, or with an —O-aryl group having 2-6 carbon atoms, wherein such alkyl or aryl group may be unsubstituted or may be substituted, e.g., with halo, hydroxy, trifluoromethyl, cyano, nitro, acyl, acyloxy, alkoxy, carboxyl, carbalkoxyl, or amino groups; or with a hydroxy, an amino or a halo group. In addition to phosphodiester linkages, the nucleotides can be connected by a synthetic linkage, i.e., inter-nucleoside linkages other than phosphodiester linkages. Examples of inter-nucleoside linkages that can be used include but are not limited to phosphodiester, alkylphosphonate, phosphorothioate, phosphorodithioate, phosphate ester, alkylphosphonothioate, phosphoramidate, carbamate, carbonate, morpholino, phosphate triester, acetamidate, carboxymethyl ester, or combinations thereof.

In another aspect, the invention provides a method for determining whether or not a test composition comprises a protein, wherein the method is performed without a wash step. This embodiment of the invention comprises a) placing a composition which comprises a detectably labeled ligand, and which may or may not comprise the protein, on a dry porous membrane; b) allowing radial migration of unbound ligand on the membrane; and c) based on the localization of the detectable label on the membrane, determining whether or not the protein was present in the test composition. In accordance with this embodiment of the invention, the test composition can be any test composition that contains or is suspected of containing a protein. Thus, the test composition that contains or is suspected of containing a protein can be a biological sample, a sample obtained from a non-biological source, such as a non-biological surface that has been swiped with a collection medium, a liquid sample obtained from, for example, a water source, a sample of a food substance, or a sample obtained from any other object or environment in which it would be desirable to determine whether a particular protein is present.

In one embodiment, the test composition which is tested for the presence or absence of a protein according to the method of the invention is a cell lysate. The cell lysate can be a lysate of any type of cells. Cell lysates can be prepared using any of the many suitable techniques that are well known to those skilled in the art. In certain embodiments, the cell lysate comprises a lysate obtained from eukaryotic cells. Thus, the cells can be obtained from an individual, such as a mammal, and tested for the expression of a particular protein that is known to bind to a particular ligand. For instance, cells can be tested for expression of a protein that is exclusively or preferentially expressed by cancer cells.

In another embodiment, the cell lysate is a prokaryotic cell lysate. The lysate may therefore be from any bacteria type. Since this embodiment permits determining protein expression of a bacterial cell lysate, it can therefore can lead to a conclusion or inference about the type of bacteria that was present in the composition tested for ligand binding according to the method of the invention. The protein could accordingly be a protein that is expressed only by certain bacterial types, and/or could be a marker of a morphological or phenotypic trait of the bacteria, such as antibiotic resistance or pathogenicity.

In another aspect, the invention provides a method for determining whether a ligand binds to one or more of a plurality of distinct proteins. This embodiment of the invention is also performed without a wash step, and it comprises: a) placing a plurality of test compositions each comprising a distinct protein and a detectably labeled ligand on separate locations of a dry porous membrane; b) allowing radial migration of unbound detectably labeled ligand at the separate locations on the membrane; and c) based on the localization of the detectable label at the separate locations on the membrane, determining whether or not the ligand binds to any one or more of the proteins on the separate locations on the membrane.

The plurality of proteins (as well as the protein(s) tested in any other aspect of the invention) can be any proteins that are known or unknown to bind to the ligand. Thus, in one embodiment, the plurality of proteins comprises a panel of distinct proteins that may or may not be related to one another and which may or may not bind to the ligand. For instance, in one non-limiting embodiment, every gene or a subset thereof in an organism can be expressed, its encoded protein isolated and purified if desired, and used in the method of the invention to determine whether or not any particular ligand binds to any of the proteins. This is useful in a variety of ways. For example, and as discussed further below, many pharmaceutical agents have so-called “off target” effects. Thus, a pharmaceutical agent that binds to a target to elicit a desirable result, but which has concomitant side-effects, could be interrogated against a panel of every expressed human protein, or a sub-combination(s) thereof. The method of the invention will demonstrate binding to the intended target protein, but will also determine binding to any other protein, and thus will be informative as to how the off-target effects could be arising.

The plurality of proteins tested using the method of the invention can comprise two or more proteins. The number of proteins tested in any particular experiment is limited only by the size of the porous substrate and the means used to detect the label, and the invention includes use of more than one membrane in series, as well as various well known high-throughput sample containers, such as multi-well assays that could be adapted to provide the dry, porous substrate. It is conceivable that the entire human proteome (approximately 35,000 genes) could be assayed to determine binding or non-binding of any detectabely labeled ligand by using the method of the invention. Thus, in various embodiments, the plurality of proteins comprises all, or a sub-combination of full-length proteins encoded by each human open reading frame (ORF). It is estimated that there are approximately 35,000 human genes, but the number of proteins is expected to be higher because of factors which include but are not necessarily limited to splice variations, post-translational processing, etc., In one embodiment, the plurality of proteins analyzed in the method of the invention comprises between 2 and 35,000 proteins, inclusive, and including all digits and ranges there between.

With respect to the protein that is applied to the dry, porous substrate, it can be provided as discussed above as a component of a cell lysate, or it can be purified. The proteins can be purified to any desired degree of purity. It can be isolated from cells that endogenously produce the proteins, or produce the proteins via genetic engineering. The protein can comprise naturally occurring amino acids or modifications thereof. The protein is not particularly limited in size or amino acid constitution, so long as it can be immobilized on the substrate. Thus, the protein can be a peptide, a polypeptide, or a protein. In various non-limiting embodiments, the protein has an amino acid length of between 10 and 35,000 amino acids, inclusive, and including integers and all ranges there between. Presently, the longest known protein is human connectin, which has a primary amino acid sequence of 34,350 amino acids and a molecular weight of approximately 3.8 megadaltons.

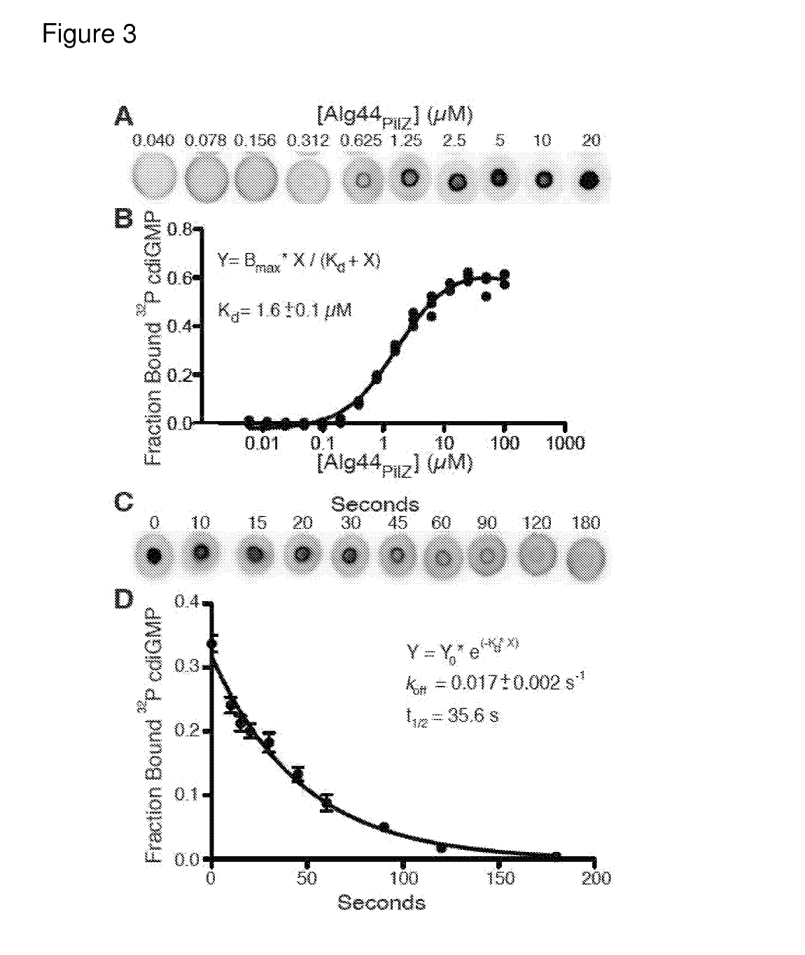

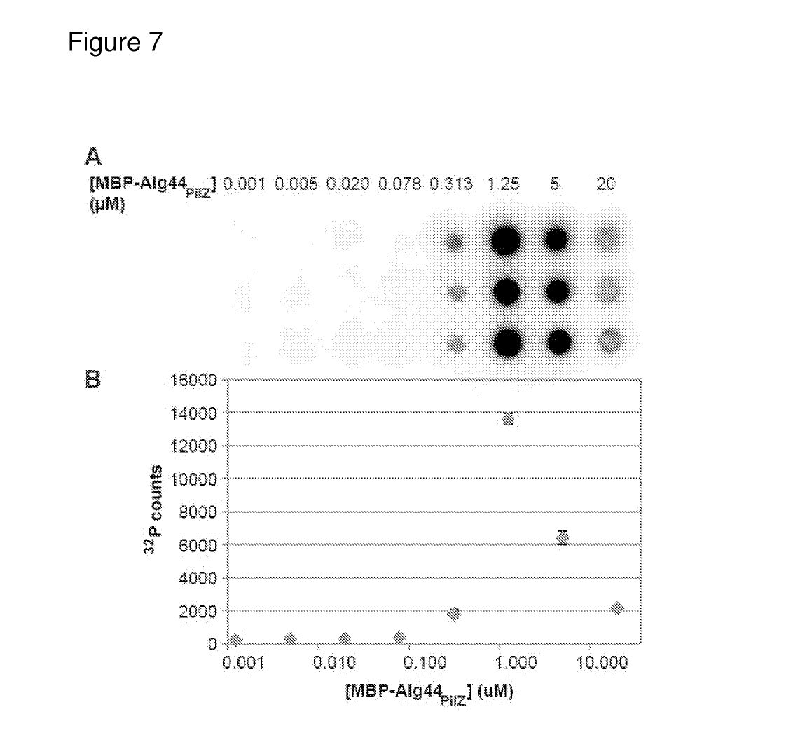

In one embodiment, the invention is practiced using a molar excess of protein, relative to the ligand. With respect to the amount of protein that is placed on the substrate, the present invention permits analysis of a range of amounts of proteins. For example, the invention includes analysis of from 1 picomole to 200 micormoles of protein, inclusive, and including all digits and all ranges there between. In a particular embodiment, from 20 micromoles to 100 micromoles of protein are used. In this regard, the elimination of a wash step for the present method permits use of larger amounts of protein that can be used by conventional filter methods because the wash step in the conventional methods tends to reduce the amount of protein that remains on the filter (see, for example, FIGS. 3A and 3B and FIG. 7).

The volume of the composition comprising the protein and/or the detectably labeled ligand can vary. In various embodiments, from 1 μl to 50 μl, inclusive, and including all digits and ranges there between, of liquid volume is used. In particular embodiments, from 1 μl to 10 μl is used.

Another advantage of the invention is the rapidity with which the assays can be performed. In various embodiments, the separation of unbound ligand from the protein by capillary action is complete in a time period of from 1 second to 90 seconds, inclusive, and including all digits and ranges there between. The speed of the assay can relate to the volume of the sample applied. For instance, in one non-limiting example, capillary action based separation of unbound ligand from protein in a 1 μl sample is complete in one second or less. In another non-limiting embodiment, capillary action based separation of unbound ligand from protein in a 10 μl sample can be complete in 30 seconds or less. Longer times can be used in certain embodiments, where for example competition between labeled and unlabeled ligands is used to analyze ligand binding parameters.

Immobilization of the protein on the porous substrate can be reversible or irreversible. The porous substrate can be any porous substrate that can facilitate capillary action based migration by the ligands used in the method of the invention. In one embodiment, the porous substrate is nitrocellulose. In another embodiment, it is diethylaminoethyl cellulose (DEAE-C). DEAE-cellulose. Any other dry porous substrate that can wick liquid from the location where the initial liquid composition is placed can be used or adapted for use in the invention. In one embodiment, the invention provides a nitrocellulose membrane comprising a plurality of locations which contain detectably labeled ligand that is bound to a protein that is immobilized on the membrane, or detectably labeled ligand that has been separated from an immobilized protein by capillary action, or a combination thereof. The nitrocellulose membranes can be prepared as such without a wash step.

A “wash” step as used herein refers to the conventional washing of substrates that are typically used for assays that involve identification of and/or separation of compounds. Those skilled in the art will recognize that wash steps are routinely employed to remove or lessen background signal that can be caused by, among other factors, non-specific binding of compounds to one another. Thus, the lack of a wash step as used herein refers to the lack of washing of the porous substrate on which the method of the invention is performed. Accordingly, performing the method of the invention without a wash step means that the porous substrate used in the method is not contacted with liquid (other than the liquid containing the protein and ligand preparations) that is intended to or does remove or reduce the amount of any particular compound from the substrate, particularly compounds that can affect ligand binding and/or ligand mobility and/or detection thereof. Accordingly, no such wash step is performed prior to determining binding of the detectably labeled ligand. The lack of a washing step as used herein is does not include contact with a liquid that is employed in the manufacturing of the porous, solid substrate used in the invention.

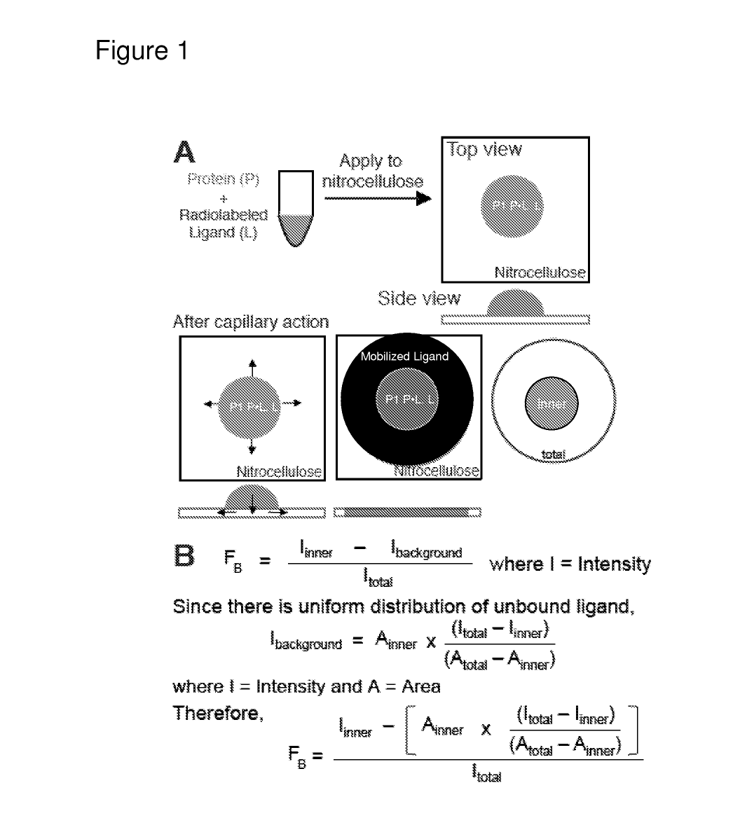

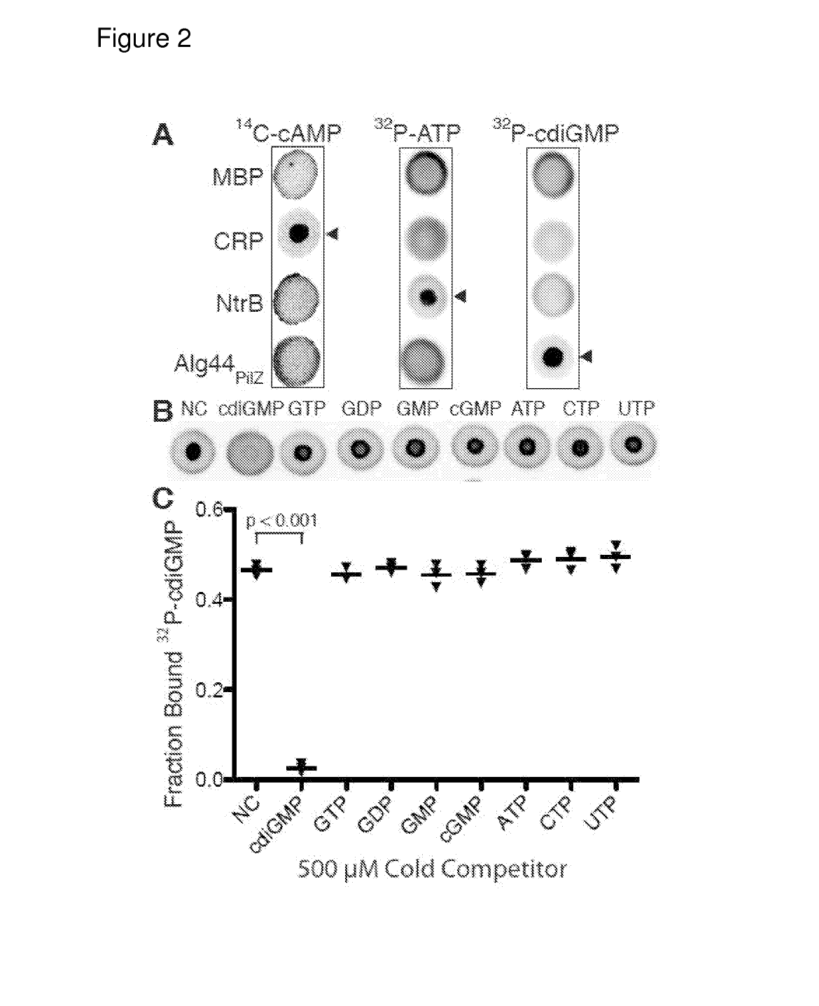

It will be recognized that, in general, various aspects of the invention relate to analysis of localization of the detectable label on the membrane. In this regard, radial migration of ligand due to capillary action results in a pattern at a location on the membrane. Since the assay depends on radial migration of unbound ligand on the membrane in an essentially horizontal plain, the pattern of ligand binding and/or mobility typically has a curved circumference. The pattern can generally resemble the geometric proportions of a circle or oval. In various aspects of the invention, the pattern produced by mixing detectably labeled ligand and protein comprises a first area where protein is immobilized on the substrate. The first area can be considered an inner area, such as an inner circle (see, for instance, FIG. 1). The inner area is encompassed within an outer area that is delineated by the location where the movement of the ligand by capillary action has stopped. Thus, the location of detectably labeled ligand that has stopped moving away from the inner area can form a circumference that is the boundary of a pattern on the substrate that contains the total area in which the detectably labeled is for any given sample. The area outside the inner area but within the total area of the pattern can be considered a second area, or an outer area. Illustrative examples of patterns produced using the method of the invention are presented in the Figures, including but not limited to FIGS. 1 and 2. The inner area is considered to comprise detectably labeled ligand that is bound to the protein and the area outside of it to comprise unbound detectably labeled ligand. The amount of unbound ligand that is present in the inner area, if any, can be calculated if desired using methods described further below, which can be of benefit when determining parameters such as the fraction of ligand bound.

In various embodiments of the invention, determining that the ligand binds to a protein at a location on the membrane can comprise determining a localization of the detectable label in an inner area of a pattern. The inner area, when detectably labeled ligand is bound to the protein, has greater signal intensity from the detectable label than the signal intensity from the remainder of the area of the pattern. Therefore, when there is little or no detectably labeled ligand bound to the protein, the pattern lacks an inner area that has greater signal intensity from the detectable label than the signal intensity from the total area of the pattern. The relationship between the signal from the inner area and the signal from the total area, with or without other parameters, can be used for various binding measurements as further described below, some of which are illustrated graphically in FIGS. 1A and 1B. For example, in one embodiment, the amount of fraction bound can be determined using the formula:

$${F}_{B} = \frac{{I}_{inner} - {A}_{inner} * ( \frac{{I}_{total} - {I}_{inner}}{{A}_{total} - {A}_{inner}} )}{{I}_{total}}$$

Some embodiments of the invention comprise multiple tests of compositions that comprise the same type of detectably labeled ligand and the same kind of protein. These include but are not necessarily limited to serial dilutions and competition assays. For example, various kinetic parameters can be determined by, for instance, addition of unlabeled ligand to compete with detectably labeled ligand so that various measurements of binding specificities and other parameters that relate to the degree of affinity of the ligand for the protein can be made (see, for example, FIG. 3). Techniques for performing and interpreting competition assays are well known in the art and can be readily adapted to be used in conjunction with the method of the invention.

In certain, non-limiting embodiments of the invention, the method can be performed for identification of ligands that have improved capacity to occupy a binding site on a protein relative to the detectably labeled ligand. For example, one or a panel of unlabeled test ligands could be used to assess the ability of the test ligand(s) to compete with the detectably labeled for binding to the protein. In one embodiment, this is performed in separate reactions by mixing increasing concentrations of the unlabeled test ligand with the detectably labeled ligand and performing the method of the invention. A test ligand which competes with the detectably labeled ligand for protein binding will lessen the intensity of the signal from the inner area of the pattern on the membrane because increasing concentrations of test ligand (and/or because or increased affinity for the protein by various test ligands) will result in less binding of the protein by the detectably labeled ligand. Thus, increasing amounts of detectably labeled ligand will be displaced from binding and will accordingly radially migrate towards the periphery of the pattern due to capillary action. This approach could be implemented to identify test ligands as, for example, pharmaceutical agents which could be used for a variety of purposes, which include but are not limited to receptor agonists and/or antagonists.

The pattern of detectably ligand localization can be detected in a variety of ways. For example, when the detectable label is a radioisotope, a system that can detect radioactive emission from the radioisotope can be used, i.e., if radiolabeled phosphorus is used, then a phosphorimager can be used to measure signal intensity and determine the pattern and respective signal intensities. Likewise, systems that employ fluorescent or colorimetric detection methods can be used when suitably labeled ligands are employed. Systems such as these can perform or assist in performing quantitative or qualitative analysis of the patterns. Additionally, visual inspection of the patterns of detectably labeled ligands by a human, whether the patterns are visualized directly or with the aid of a system, can provide qualitative determinations of ligand protein binding.

In connection with the ligand label, as discussed above, any detectable ligand can be used. The ligand can be radiolabeled (i.e., with isotopes of phosphorus, hydrogen, carbon, sulfer, nitrogen, etc.), or fluorescently labeled (fluorescein isothiocyanate (FITC), tetramethylrhodamine isothiocyanate (TRITC), etc), or labeled with a ligand that can be detected colorimetrically (choromophore o-nitrophenyl (ONP)).

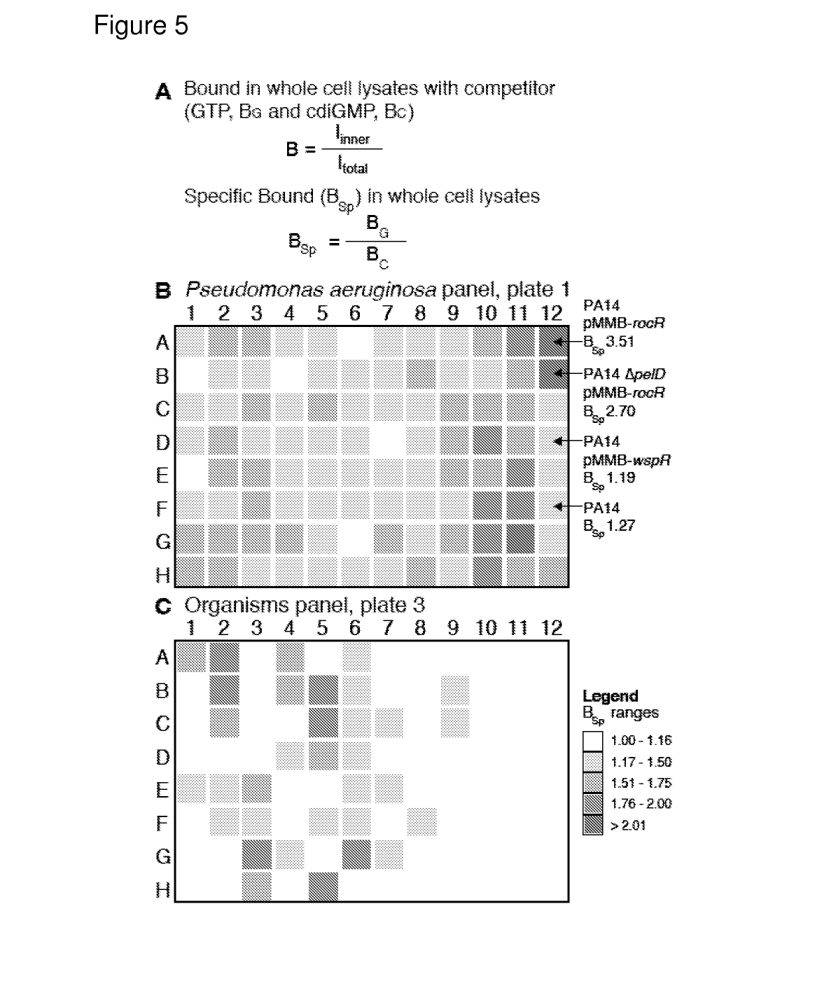

It will be apparent to the skilled artisan from the foregoing that the principle of DRaCALA should be universally applicable to any system in which the ligand can be mobilized by capillary action in conjunction with a solid support capable of sequestering the macromolecule. Thus, the choice of the support, the solvent composition of the mobile phase and the specific properties of the ligand can be altered to enhance the effectiveness of DRaCALA for various protein-ligand systems. In this regard, studies of numerous systems involving low molecular weight biological ligands and receptor macromolecules can benefit from DRaCALA including but not necessarily limited to nucleotide derivatives, amino acid derivatives, metal ions, sugars and other small signaling molecules. The function of biological ligands can be specific to subset of organisms that produce or utilize these molecules. To identify biological samples that may be enriched in the ligand binding protein, a similar approach to the screen for cdiGMP binding proteins (FIG. 5C) can be taken to identify model organisms for the study of ligands. Alternatively, expression of ligand-binding proteins may be regulated. DRaCALA provide a method for rapidly screening a single organism grown in different conditions, such the phenotypic arrays, and test for ligand binding activity.

The systematic identification of protein receptors for each of these small molecular ligands will allow for a comprehensive understanding of the biological effect of these signaling molecules and DRaCALA offers a high throughput platform that should greatly facilitate this process. Furthermore, the ability of DRaCALA to detect ligand binding in whole cell extracts should allow for systematic screening of whole-genome open reading frame libraries (ORFeomes) for proteins that bind various small molecules. DRaCALA is scalable, and has been performed using both a standard single channel pipette and an 8-channel multichannel pipette with equal precision and accuracy. Thus, DRaCALA could be easily adapted for high-throughput applications by, in various embodiments, using a 96-well pin tool in combination with standard robotics. The volume required for the DRaCALA assay can be further reduced to allow for screening using the 384-well format. An important advantage of DRaCALA is that insoluble proteins appear to have a similar behavior as soluble protein in whole cell lysates, thus avoiding purification problems associated with insoluble proteins.

Development of DRaCALA as a high-throughput assay for the detection of protein-ligand interactions will be useful for identifying new targets for pharmaceutical intervention. To this end, DRaCALA might be used initially as a screening tool to identify new interaction pairs, and then in a second round of DRaCALA to identify inhibitors that prevent the interaction. Labeling of the identified specific inhibitor would then allow for rapid sequential screening for even more potent molecules that can displace the original inhibitor. Our results therefore show that DRaCALA can be developed as a platform to enable critical advances in metabolite interactomics and therapeutic intervention.

### Example 1

This Example provides a description of various non-limiting embodiments of the invention which demonstrate determining detectably labeled ligand binding to proteins.

### Principle of Differential DRaCALA

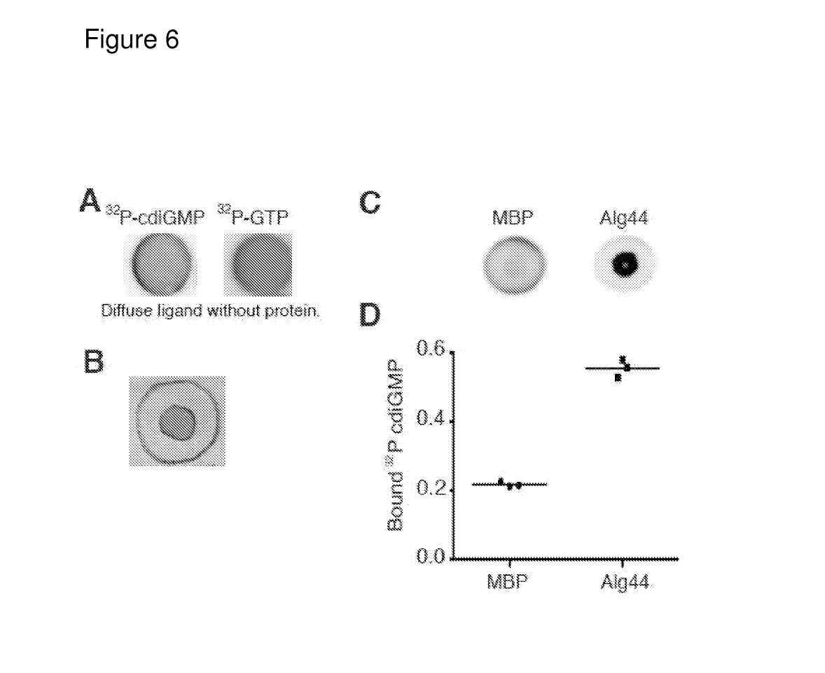

DRaCALA exploits the ability of nitrocellulose membranes to preferentially sequester proteins over small molecule ligands. When a mixture of protein and radiolabeled ligand is spotted onto a dry nitrocellulose membrane, protein and bound ligand are immobilized at the site of contact while free ligand is mobilized by capillary action with the liquid phase (FIG. 1A). DRaCALA is a rapid assay, as the capillary action can be completed in less than 5 seconds. Since DRaCALA does not utilize a wash step, the pattern of ligand migration allows a rapid detection of both the total ligand and the ligand sequestered by proteins. Because capillary action distributes the unbound ligand throughout the mobile phase, the calculation for the fraction bound (F_{B}) can be corrected for this background (see below for edge effects at the solvent front and annulation of the protein). Therefore, F_{B} is defined by the equation in FIG. 1B, where Iᵢₙₙₑᵣ is the intensity of signal in the area with protein (inner circle) and Iₜₒₜₐₗ is the total signal of the entire sample (outer circle). The Iᵢₙₙₑᵣ signal consists of both ligand bound to protein and unbound ligand that has not mobilized beyond the area of the inner circle, which we define as I_{background}. I_{background} can be calculated by subtracting the signal intensity of the Iᵢₙₙₑᵣ from the total ligand Iₜₒₜₐₗ and adjusting for the relative areas of the inner (Aᵢₙₙₑᵣ) and outer circles (Aₜₒₜₐₗ) (FIG. 1B). For free ligand alone, the signal for the ligand is not inner, sequestered and therefore has a baseline F_{B} of 0.01±0.04 (FIG. 6A), whereas protein alone does not mobilize on nitrocellulose (FIG. 6B).

### DRaCALA Detection of Protein-Ligand Interactions

The principle of DRaCALA was illustrated by measuring ligand binding to known nucleotide binding proteins: P. aeruginosa Alg44_{PilZ} binds cyclic-di-GMP (cdiGMP), E. coli CRP binds cAMP, and E. coli NtrB binds ATP. Radiolabeled ligands were incubated with each of the proteins and the mixtures were spotted on nitrocellulose. After spreading by capillary action, membranes were dried and quantitated by phosphorimager. Maltose binding protein (MBP), which does not bind to any of these small molecules, was used as a control. Each of the radiolabel signals from MBP mixtures was distributed by capillary action (FIG. 2A). CRP specifically bound cAMP, as demonstrated by the sequestration of the signal, but it did not bind cdiGMP or ATP (FIG. 1A). NtrB bound ATP, but not cdiGMP or cAMP (FIG. 2A). Similarly, Alg44_{PilZ} bound cdiGMP, but not cAMP or ATP (FIG. 2A). The specificity of Alg44_{PilZ} binding to cdiGMP was further tested by competition with excess unlabeled nucleotides (400-fold molar excess relative to the Alg44_{PilZ} protein). Alg44_{PilZ} binding to ³²P-cdiGMP was abolished by cdiGMP, but not by cGMP, GMP, GDP, GTP, ATP, CTP or UTP as was previously described (FIG. 2B). The F_{B} for cdiGMP was 0.31±0.07, which was reversed by competition with unlabeled cdiGMP to the background level of 0.04±0.01 (FIG. 2C).

### Use of DRaCALA to Quantitate Protein-Ligand Interactions

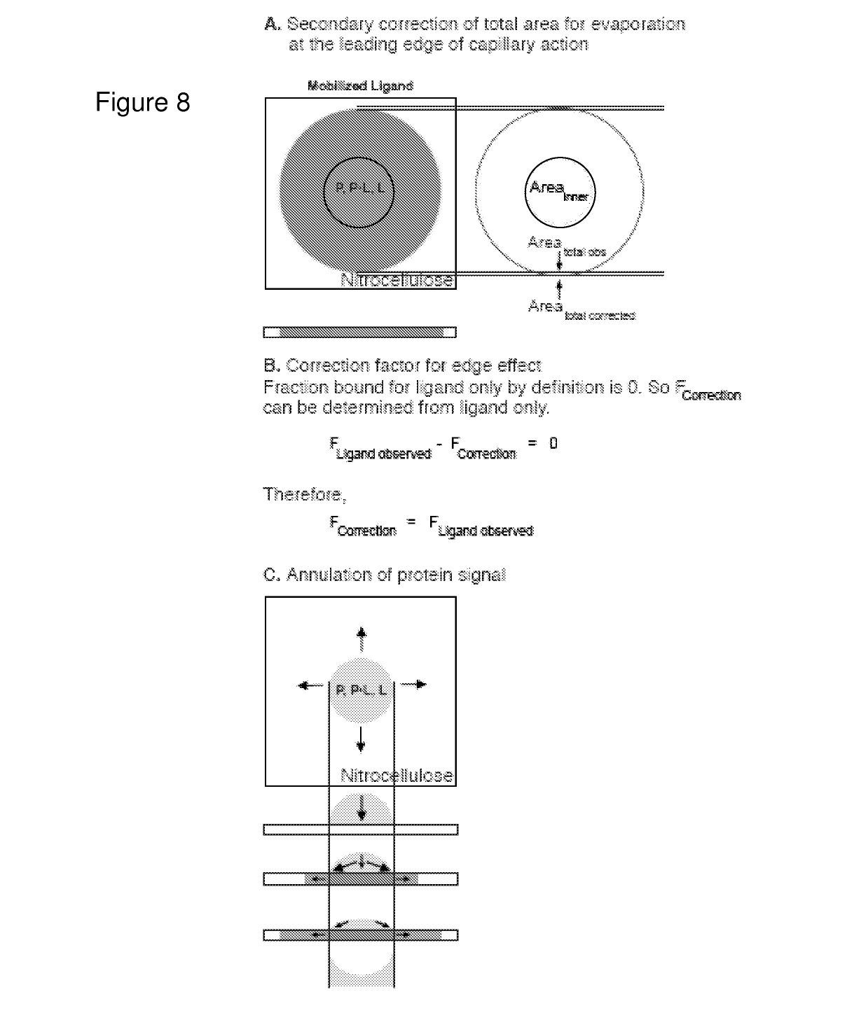

In addition to qualitative assessments of specific protein-ligand interactions,
DRaCALA is useful for quantitating biochemical parameters, including the dissociation constant (K_{d}) and the dissociation rate (k_{off}). K_{d} can be measured by altering either the protein or ligand concentrations in titration experiments; since DRaCALA detects only the ligand mobility, ligand concentrations can be always held constant. As an example, the K_{d} of Alg44_{PilZ} binding to cdiGMP was determined by analyzing mixtures of 4 nM ³²P-cdiGMP with 0.006-100 μM Alg44_{PilZ} (FIG. 3A). At concentrations of protein above the K_{d}, the F_{B} approaches saturation; this binding decreases as the Alg44_{PilZ} concentration is decreased, reaching a level indistinguishable from background at the lowest protein concentrations. Analysis of this binding curve indicated a K_{d}=1.6±0.1 μM, which is in reasonable agreement with the previously determined value of 5.6 μM determined by ITC (FIG. 3B) (Merighi M, Lee V T, Hyodo M, Hayakawa Y, & Lory S (2007) Mol Microbiol 65:876-895). Application of identical samples to the dot blot apparatus for vacuum-mediated filter binding assay resulted in problems associated with high protein concentrations and, as a consequence, difficulty in assessing saturation of binding (FIG. 7). Since the assay is completed in less than 5 seconds, DRaCALA can also be used to determine the k_{off} for those protein-ligand complexes with slower off rates. k_{off} was determined for cdiGMP and Alg44_{PilZ} by spotting at the indicated time points after the addition of 1 mM unlabeled cdiGMP to a pre-incubated mixture of Alg44_{PilZ} and ³²P-cdiGMP (FIG. 3C). The fractions bound were plotted against time and analyzed by non-linear regression, which yielded a k_{off} of 0.017±0.002 sec⁻¹ corresponding to a half-life (t_{1/2}) of 35.6±10.7 seconds (FIG. 3D). The binding of ³²P cdiGMP to Alg44_{PilZ} was completely competed away by 1 mM unlabeled cdiGMP within 90 seconds. Occasionally, we observed an increased signal at the leading edge of the capillary action and in the protein portion of the DRaCALA spot. Both edge and annulation effects are explained in FIG. 8. The edge effect is due to evaporation of the solvent during the time of the experiment, and is dependent on the humidity of the local environment around the nitrocellulose support. The evaporation results in a smaller total area (A_{total observed} in FIG. 8A) and leads to an increased value in the calculated I_{background}. As a secondary correction for the edge effect, the fraction bound determined for spotted ligand in the absence of protein can be subtracted from all samples in parallel (FIG. 8B). The annulation effect does not alter the F_{B} calculation (FIG. 8C). Results from these experiments demonstrate the utility of DRaCALA for rapid and precise quantitation of biochemical parameters.

### DRaCALA Detection of Ligand-Binding Proteins in Whole Cells

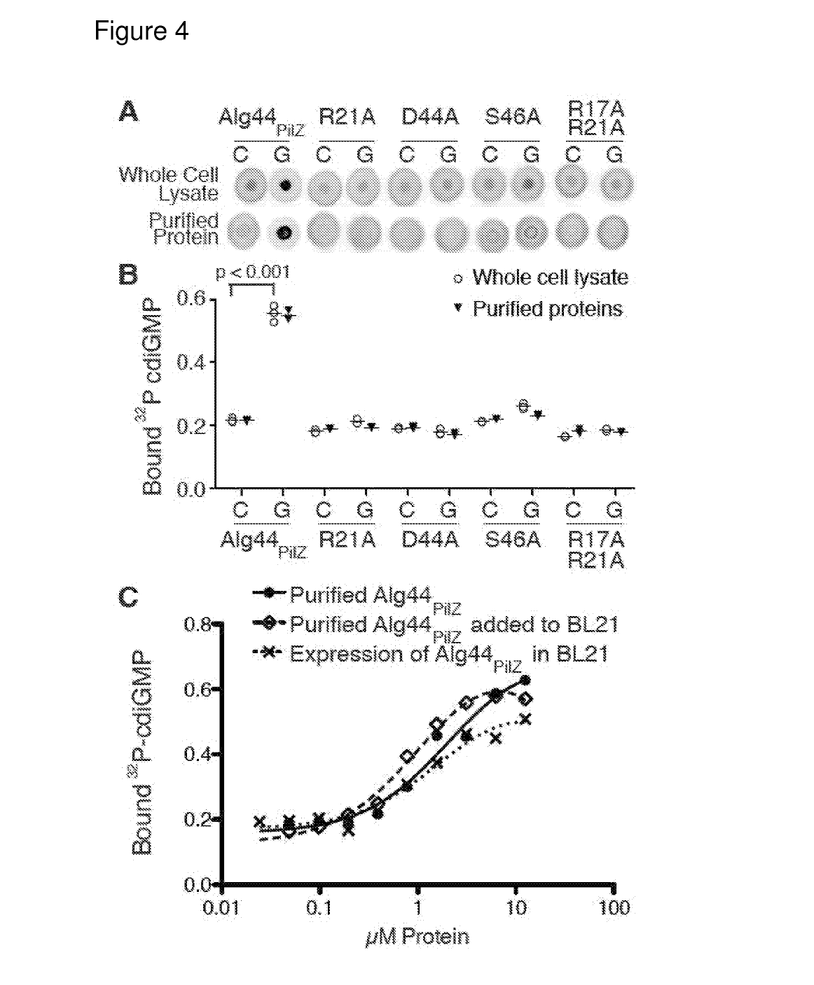

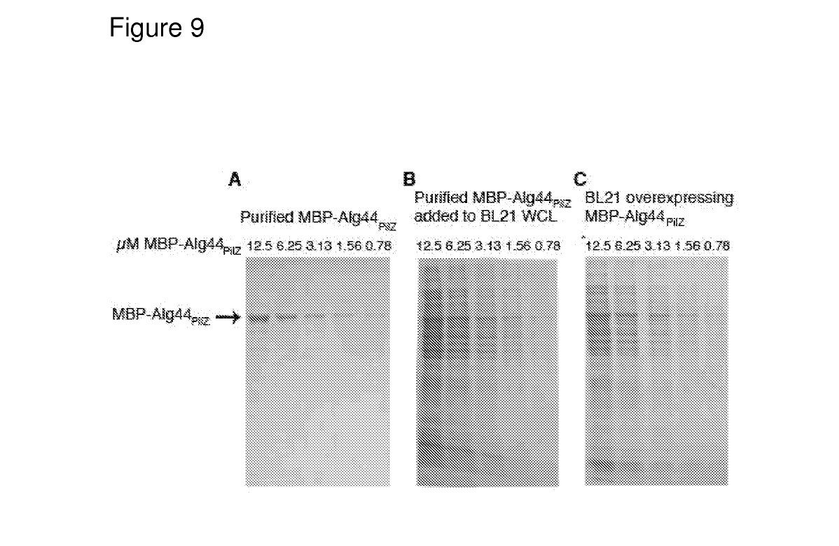

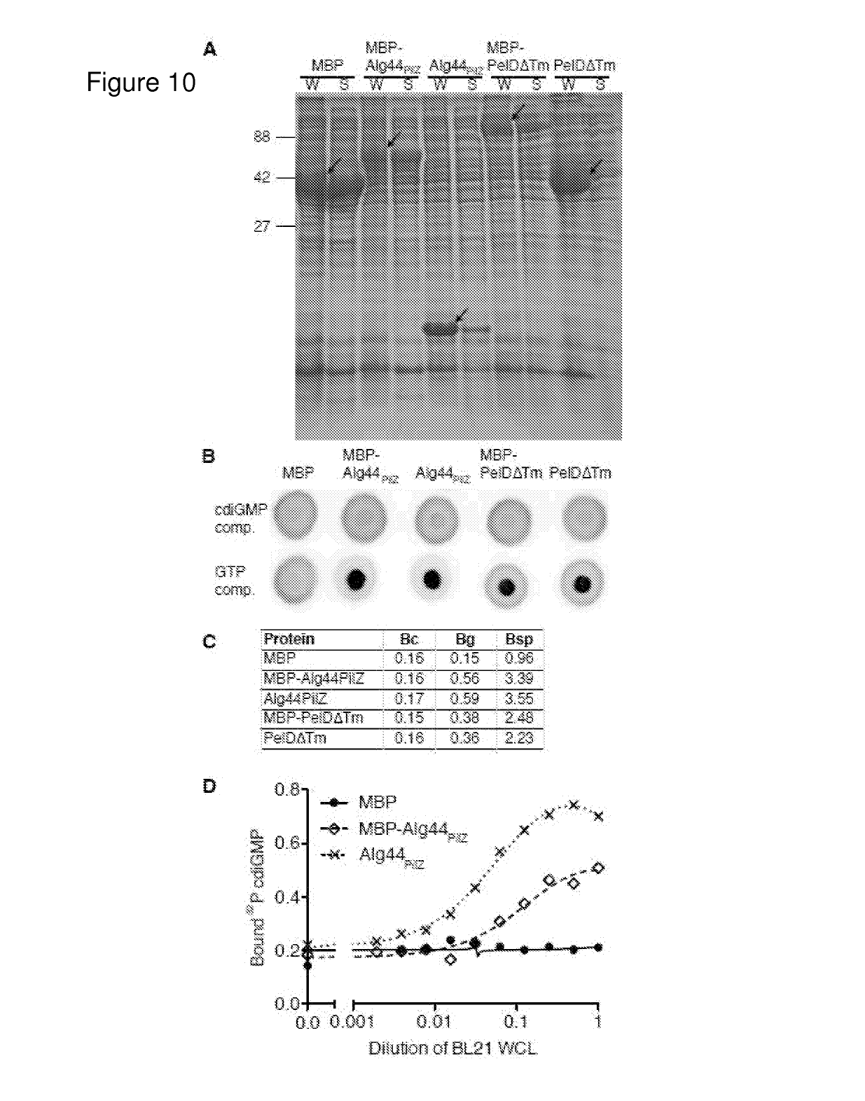

A major limitation of most biochemical assays is the requirement for purified protein. We asked whether DRaCALA could be applied to crude extracts to overcome this limitation. Alg44_{PilZ} binding to cdiGMP requires a number of conserved residues, including R17, R21, D44 and S46, in the PilZ domain of the protein. E. coli BL21(DE3) expressing Alg44_{PilZ} and variants with R21A, D44A, S46A, and R17A/R21A substitutions were lysed and tested for binding to cdiGMP using DRaCALA. Protein extracts from E. coli expressing MBP alone did not bind cdiGMP (FIGS. 6C and 6D). Only the whole cell lysates from E. coli expressing wild-type Alg44_{PilZ} sequestered ³²P-cdiGMP (FIG. 4A). Specificity of ³²P-cdiGMP in the background of all other cellular macromolecules was demonstrated by competition with 1 mM of unlabeled specific competitor cdiGMP or the non-specific competitor GTP. A significant difference between the bound fractions for cdiGMP or GTP competition experiments was detected for the wild-type Alg44_{PilZ}, but not for the PilZ domain mutants (FIGS. 4A and 4B). The results from whole cell lysates are in agreement with the results obtained with purified proteins (FIG. 4B). The sensitivity of DRaCALA detection of MBP-Alg44_{PilZ} binding to cdiGMP was tested by testing serial dilutions of purified protein alone or in the presence of BL21(DE3) whole cell lysates. The results show that the binding of cdiGMP by MBP-Alg44_{PilZ} is not affected by the presence of cellular proteins (FIG. 4C). Furthermore, serial dilution of extracts from BL21(DE3) cells expressing MBP-Alg44_{PilZ} also resulted in a similar binding curve for comparable levels of MBP-Alg44_{PilZ} proteins (FIG. 4C and FIG. 9). A common problem during expression of heterologous protein in a foreign host is that the protein is often insoluble and forms inclusion bodies. Expression of both Alg44_{PilZ} and PelD without the MBP tag resulted in insoluble proteins (FIG. 10A). We tested the whole cell extracts with soluble and insoluble proteins and found that either form of the protein can specifically sequester cdiGMP by DRaCALA (FIGS. 10B and 10C). Serial dilution of the whole cell extracts reduced cdiGMP sequestration to background levels for BL21 whole cell extracts (FIG. 10D). The ability to detect protein-ligand interactions in whole cell lysates makes DRaCALA amenable to high-throughput analysis of whole cell lysates for the presence of ligand-binding proteins.

DRaCALA Detection of cdiGMP Binding Proteins in Diverse Prokaryotic and Eukaryotic Organisms.

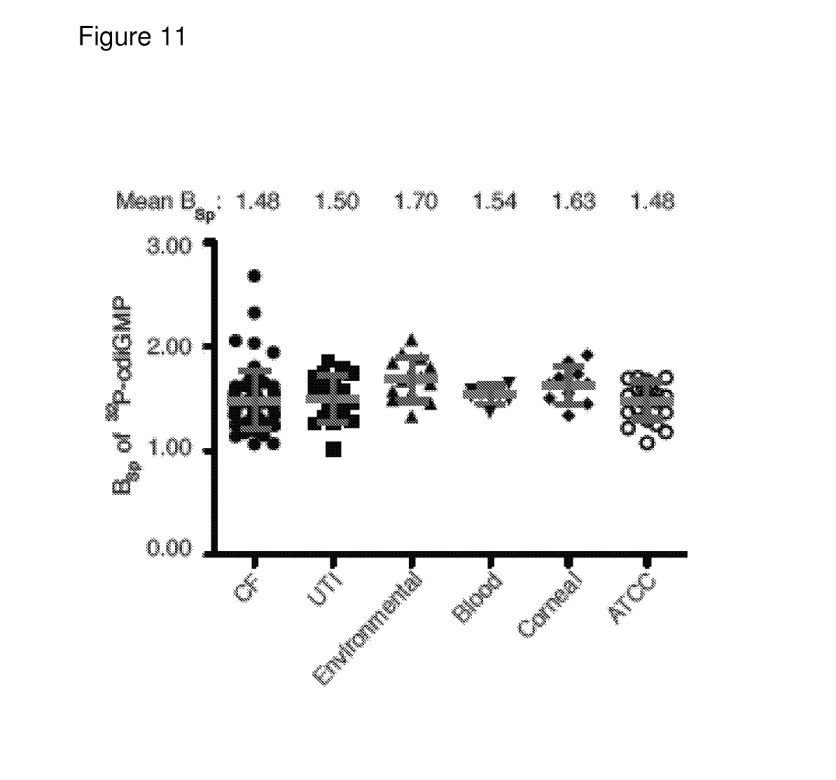

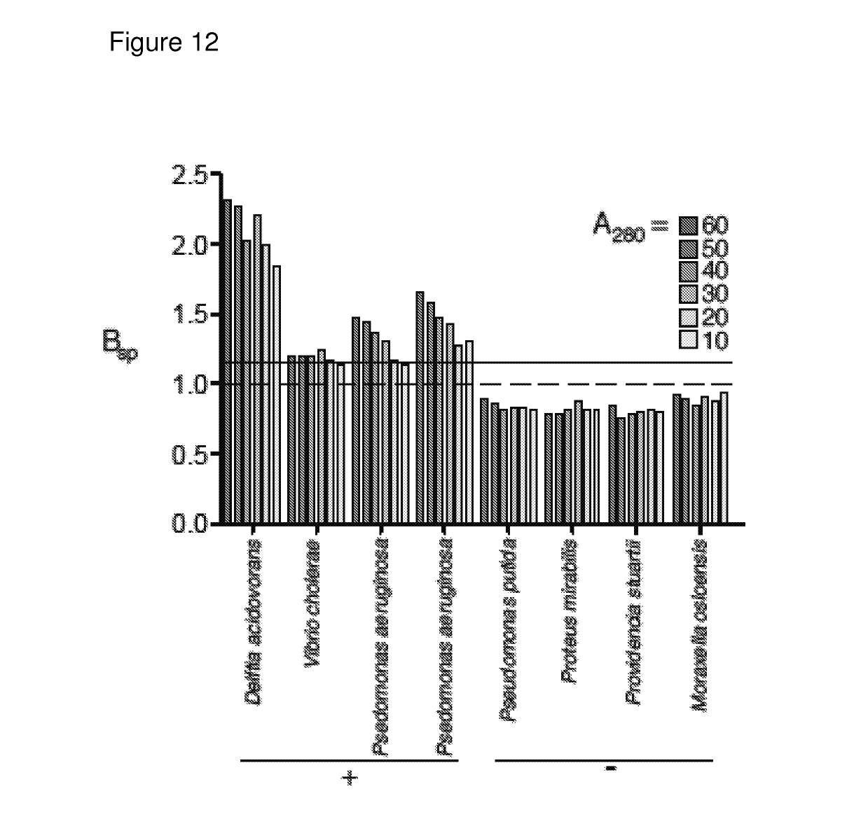

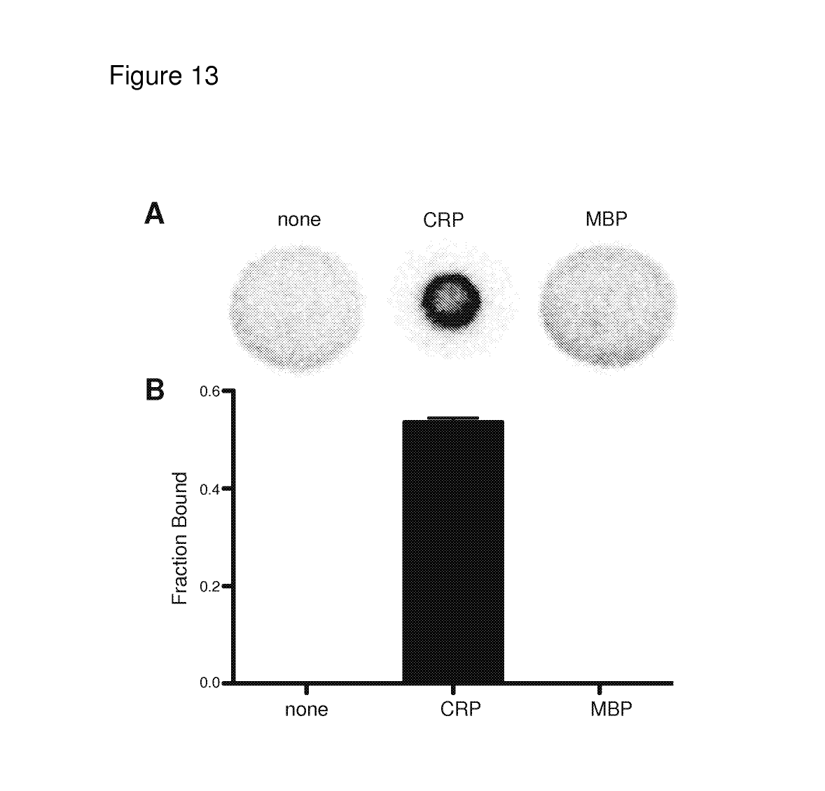

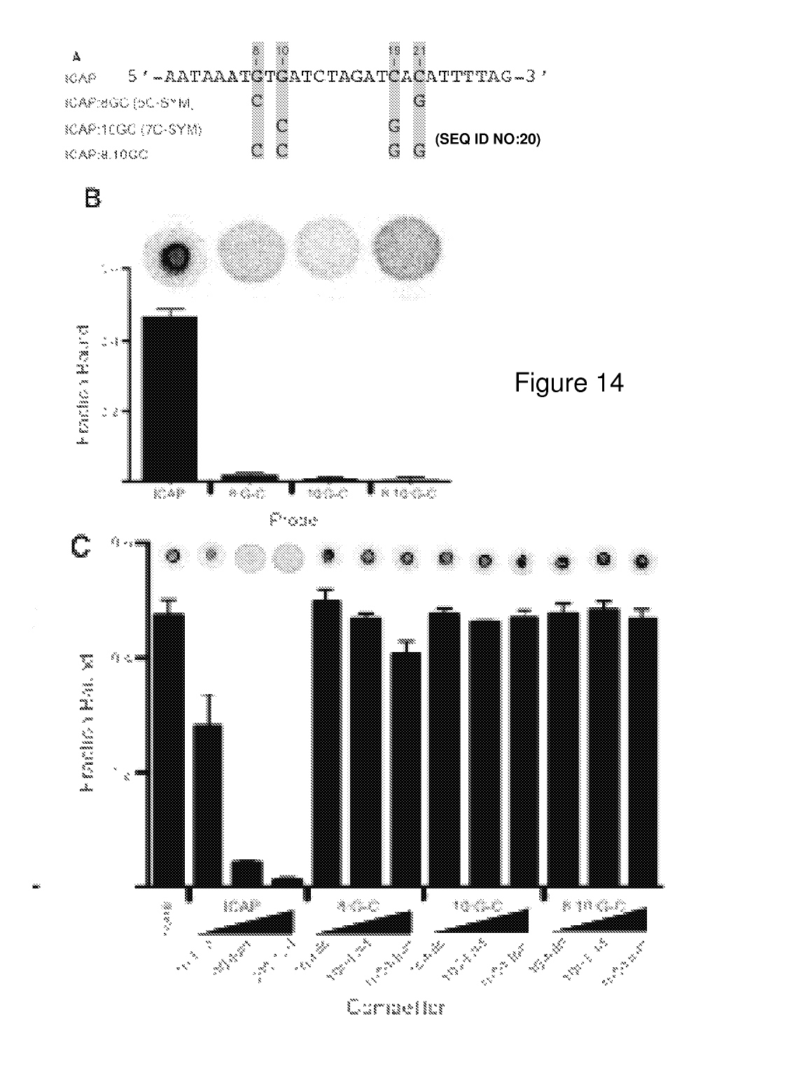

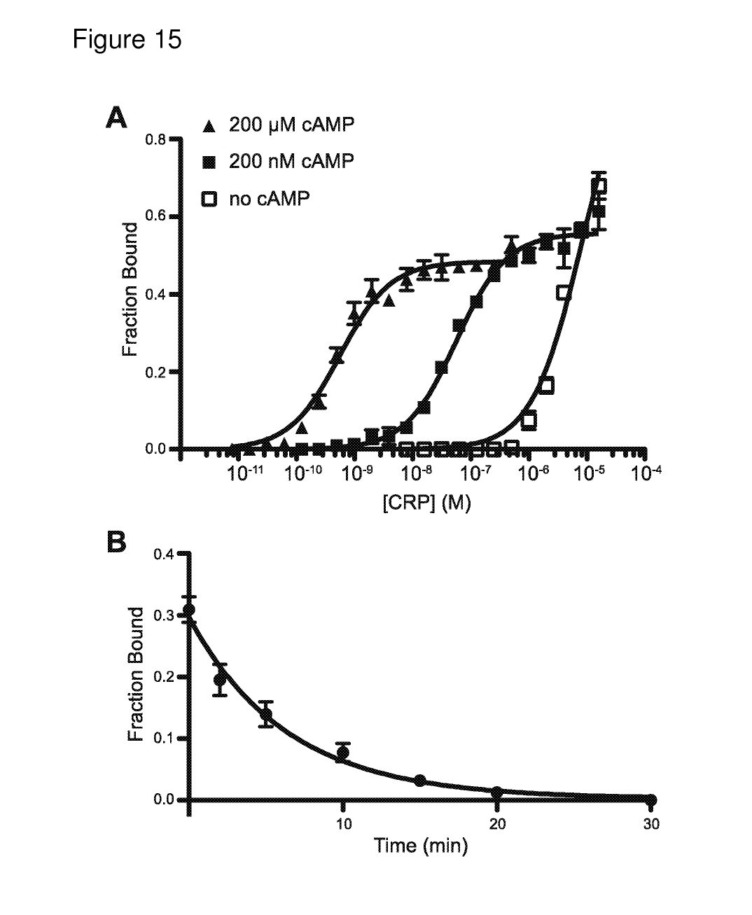

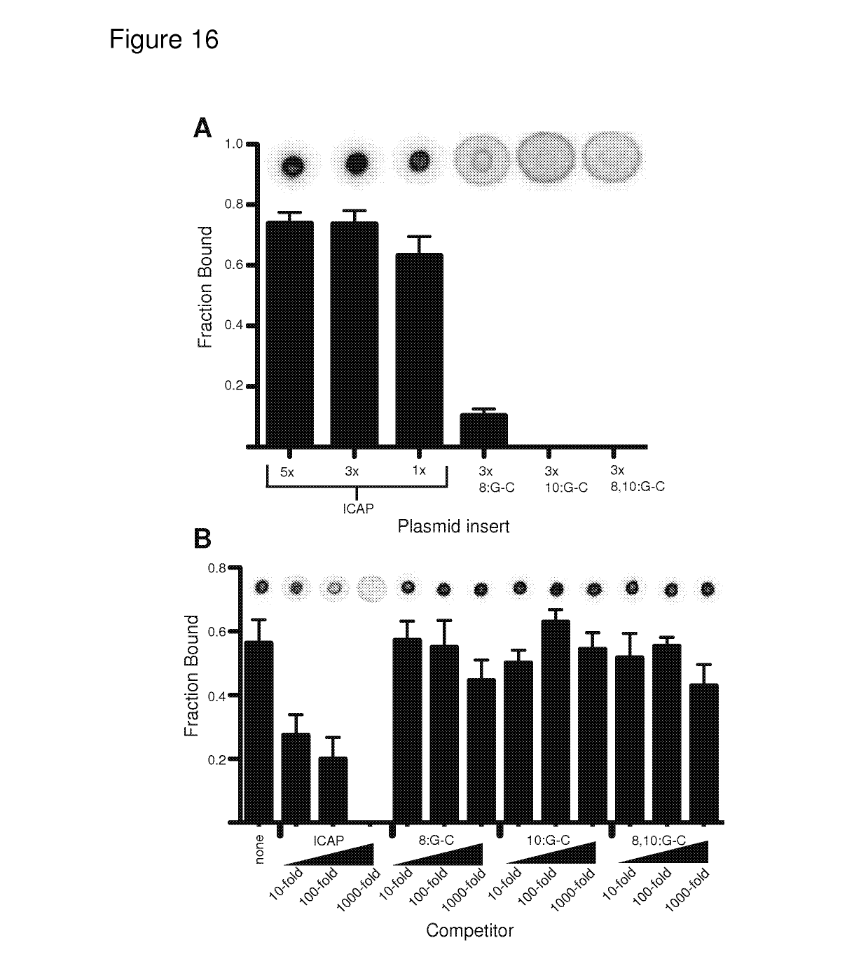

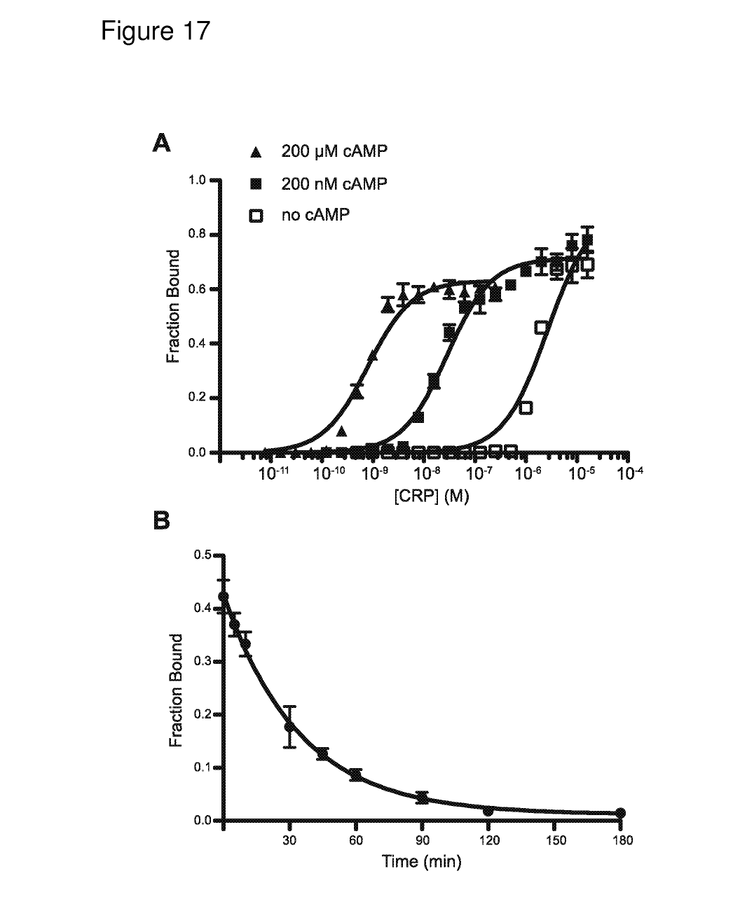

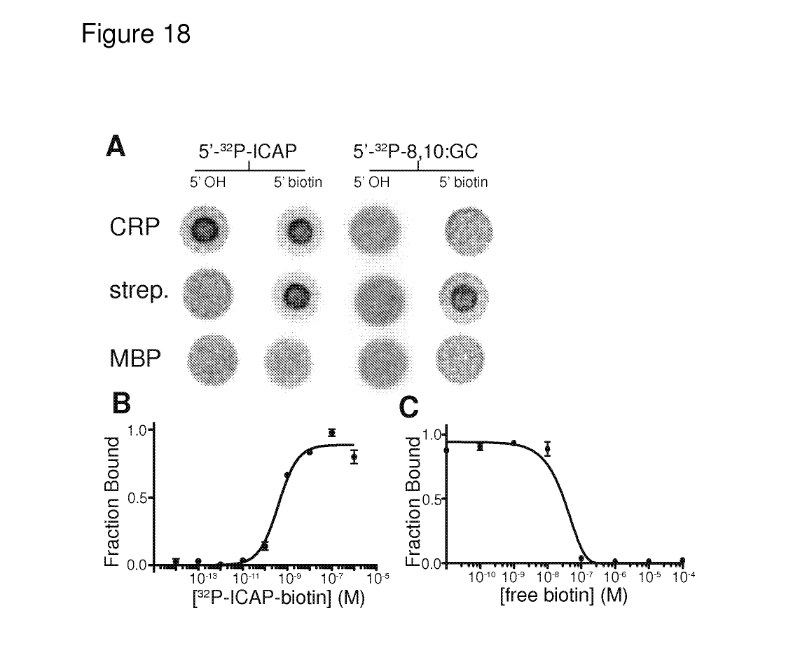

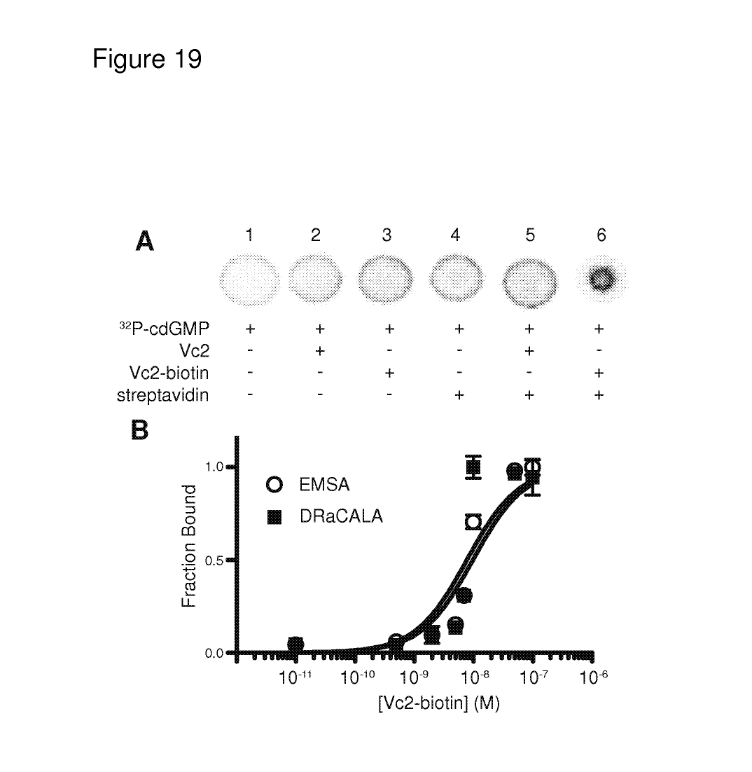

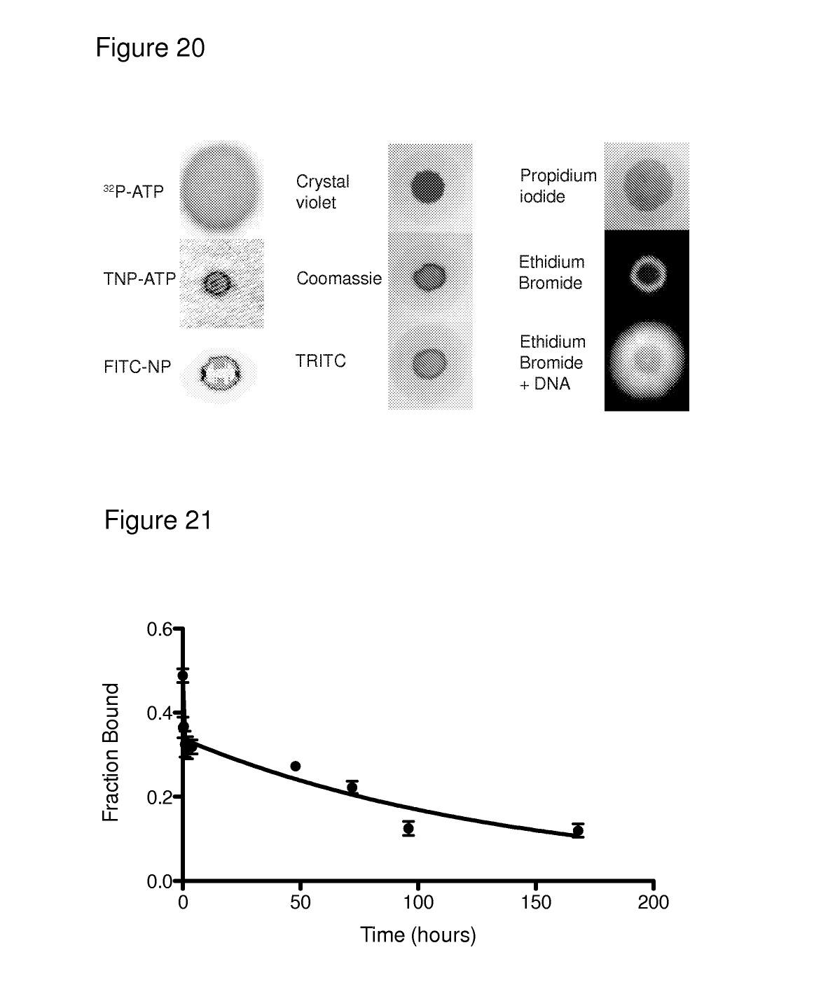

The applicability of DRaCALA to high-throughput metabolite interactomics was demonstrated by screening for binding proteins of an important secondary signaling dinucleotide, cdiGMP. Recent findings have identified cdiGMP as the signaling molecule that controls biofilm formation, motility and a number of other bacterial functions. Although the enzymes known to synthesize and degrade cdiGMP are restricted to bacteria, there are questions as to which bacterial species express cdiGMP-binding proteins. cdiGMP has also proven to be useful as an adjuvant during immunization to enhance the mammalian immune response, which suggests that there may be cdiGMP-binding proteins in higher eukaryotes. We used DRaCALA to test 191 strains of P. aeruginosa and a panel of 61 other species in a 96-well plate format. As a control for specificity, each extract was tested for binding to the labeled ligand by competition with the unlabeled specific or non-specific ligand. As in the example of whole cell lysates of E. coli, unlabeled GTP competitor was used to detect specific ³²P-cdiGMP binding (bound during GTP competition or B_{G}), and unlabeled cdiGMP competitor was used to detect non-specific ³²P-cdiGMP binding (bound during cdiGMP competition or B_{C}) (FIG. 5A). The ratio of B_{G} to B_{C} is called specific binding (B_{Sp}). The limit of non-specific binding was calculated by adding 2 standard deviations to the average B_{C} resulting in a conservative cutoff value for positive B_{Sp} of 1.17 (FIG. 5A and Table 6). Of the 191 P. aeruginosa isolated from various sources, 184 (96%) displayed a positive B_{Sp} value greater than 1.17 (96 samples shown in FIG. 5B and all data presented in Table 6). These results suggest that most P. aeruginosa strains express detectable levels of cdiGMP-binding proteins. When strains isolated from different sources were analyzed for cdiGMP binding, all groups had an average B_{Sp} value greater than 1.48 suggesting that cdiGMP signaling is retained (FIG. 11 and Table 6). The range of cell lysate concentrations required for consistent signal detection was tested by diluting cell extracts to 10 to 60 absorbance (280 nm) units in intervals of 10. Each lysate dilution yielded similar B_{Sp} values suggesting that this range of cell lysate concentration provides a reliable readout for the detection of c-di-GMP binding (FIG. 12).

One potential complicating factor is the effect of cdiGMP metabolism and endogenous cdiGMP levels on the DRaCALA readout. Extracts of the laboratory P. aeruginosa strain PA14 overexpressing either the phosphodiesterase (PDE) RocR or the diguanylate cyclase (DGC) WspR were tested for their ability to bind cdiGMP. Wild-type PA14 showed B_{Sp} of 1.27 indicating that cdiGMP-binding proteins are not fully occupied by endogenous cdiGMP consistent with what is expected for signaling systems (F12 of plate 1 of FIG. 5B and Table 6). Increasing the cellular cdiGMP concentration through WspR overexpression decreased B_{Sp} to 1.19 (D12 of FIG. 5B and Table 6). Reducing cellular cdiGMP through RocR overexpression increased B_{Sp} to 3.51 (A12 of FIG. 5B and Table 6). The amount of cdiGMP sequestered by a whole cell extract should reflect the amount and affinity of the binding proteins present. This was tested by overexpression of RocR in the PA14ΔpelD background, which lacks the cdiGMP-binding protein PelD. Without PelD, the B_{Sp} was reduced to 2.70 (B 12 of FIG. 5B and Table 6), indicating that PelD is an important binding protein for cdiGMP and that other proteins also bind cdiGMP. These results indicate that endogenous cdiGMP metabolism affects, but does not abolish, the ability of DRaCALA to detect cdiGMP binding proteins.

cdiGMP signaling occurs in a wide variety of bacterial species, but is not known to be present in Eukarya. We tested 54 bacterial species from 37 genus and 7 eukaryotic species including protozoa, fungi, nematodes, plants and mammals. Of the 82 tested bacteria strains, 31 (38%) displayed a B_{Sp} greater than 1.17. Included in the 31 positive samples are 21 species for which functional cdiGMP signaling has yet to be demonstrated, of which four species, Serratia marcescens, Pseudomonas alcaligenes, Pseudomonas diminuta, and Brevundimonas vesicularis, have not yet been sequenced (FIG. 5C and Table 7). We tested six bacterial species with sequenced genomes but do not have annotated DGCs and all of them failed to sequester cdiGMP above the threshold. We also tested eight eukaryotic species, none of which have annotated DGCs. Whole cell extracts of protozoa, fungi and nematodes displayed B_{Sp} below 1.17, indicating that cdiGMP-binding proteins are absent or below the limit of detection. Mammalian tissue extracts from rodent and human cell lines displayed high non-specific binding with B_{C} values greater than three standard deviations above the average B_{C} (>0.233, F6, E12, G12 and H12 in FIG. 5C and Table 7). Furthermore, the non-specific binding was eliminated after three 2-fold dilutions of these tissue extracts, indicating that mammalian tissues may contain receptors with low affinity or low abundance. Only positive B_{Sp} results from DRaCALA can be interpreted for utilization of cdiGMP signaling. As a result, DRaCALA is most effective in whole cell extracts with a low non-specific binding. Utilization of DRaCALA in a high-throughput format has expanded our knowledge of the bacterial organisms harboring cdiGMP-binding proteins and confirmed the absence of abundant high-affinity cdiGMP binding proteins in eukaryotes.

### Example 2

This Example demonstrates illustrative embodiments of the invention whereby protein-polynucleotide binding can be determined.

### DNA Oligonucleotides are Mobile in DRaCALA and Sequestered by Protein Binding

Double stranded mobility on nitrocellulose was tested using 5′-end labeled duplex DNA formed by annealing a pair of 40 bp oligonucleotides that generate the CRP consensus binding site, ICAP (gd126 and gd127 in Table 10). When the ³²P-labeled DNA was spotted on dry nitrocellulose, the ³²P radiolabel was mobilized by radial capillary action resulting in a homogenous signal across the total sample area (FIG. 13A) similar to results obtained for cAMP and ATP as described above. Addition of 100 nM CRP and 200 μM cAMP to the ICAP probe is known to promote DNA-protein complexes. Spotting of the CRP-ICAP mixture at equilibrium resulted in sequestration of the soluble probe by the immobilized protein. Maltose binding protein (MBP), which does not bind DNA, did not sequester the probe, resulting in a uniform distribution of the radiolabel as in the control without any protein. This shows that specific molecular interaction is required for probe sequestration. Quantification of the fraction bound revealed that probe alone and probe mixed with non-specific protein have no fraction bound (FIG. 13B). These results demonstrate the ability of DRaCALA to detect interactions between proteins and double stranded DNA.

### Oligonucleotide-Protein Interactions are Specific in DRaCALA

CRP interaction with ICAP requires sequence-specific inverted repeats. To test if DRaCALA can detect changes in DNA-protein interaction with single base pair changes, point mutants were generated in the ICAP site at positions that are known to abolish binding. Specifically, the guanosines at position 8 and position 10 were changed to cytosines. Because the site is symmetrical, the corresponding cytosines at positions 19 and 21 were changed to guanosines (FIG. 14A). These various probes were tested, at 4 nM, for binding to CRP by DRaCALA. The wild-type ICAP was sequestered by 100 nM CRP as before. The 8:GC mutant (G to C at position 8 and C to G at position 21) showed a very low level of binding to CRP while the 10:GC mutant (G to C at 10 and C to G at 19) and the 8,10:GC double mutant exhibited no binding (FIG. 14B). To confirm specificity, the binding between wild-type ICAP and 100 nM CRP was subjected to competition by wild-type and mutant unlabelled DNA at 10, 100, or 1000 times the concentration of the labeled DNA. The wild-type competitor partially competed at 10-fold excess and competed more significantly with increased amount of competitor (FIG. 15C). The 8:GC competitor showed no competition at 10- or 100-fold excess but did display some minor competition at 1000-fold. The 10:GC and 8,10:GC failed to compete regardless of their concentration. These results collectively show that DRaCALA measures sequence-specific DNA binding.

### DNA-Binding Affinity and Kinetics can be Measured by DRaCALA

In order to accurately describe the activity of a transcription factor or other protein on a DNA binding site, it is desirable to determine the affinity and kinetics of the DNA-protein interaction. Because radionuclides can be detected with high sensitivity, DRaCALA can be used to make such measurements for high affinity interactions. Serial two-fold dilutions of CRP were mixed with limiting ³²P-labeled ICAP probe (5 pM) to find the affinity of CRP for ICAP. CRP bound ICAP with maximum affinity when it was saturated with 200 μM cAMP. Analysis of these results indicated a dissociation constant (K_{d}) of 5.6±0.46×10⁻¹⁰ M (S.D.) (FIG. 16A). This is consistent with previously reported values for ICAP (5) (Table 9). In the absence of cAMP, the affinity of CRP for ICAP was ten thousand-fold lower (K_{d}=8.39±1.19×10⁻⁶ M (S.D.)). In the presence of an intermediate level of cAMP (200 nM) that wasn't expected to saturate the allosteric cAMP-binding site in CRP, we observed an intermediate affinity for the CRP-ICAP binding interaction (K_{d}=5.6±0.38×10⁻⁸)

We also used the CRP-ICAP binding interaction to test whether DRaCALA can be used to easily monitor the dissociation kinetics for protein-DNA complexes. A limiting amount of ³²P-labeled ICAP (5 pM) was mixed with a protein concentration just above the K_{d} (5 nM). Then, unlabeled competitor ICAP was added in 1000-fold excess of radiolabeled ligand and spots were made over time, and these spots were analyzed to monitor the fraction of ICAP bound as a function of time. Our analysis indicated a dissociation rate (k_{off}) of 2.6±0.40×10⁻³ s⁻¹ (S.D.) for the CRP-cAMP complex, corresponding to a half-life of 4.42 minutes (FIG. 15B). Using the DRaCALA-observed off-rate and affinity, the calculated on-rate is kₒₙ=4.7×10⁶ M⁻¹s⁻¹. These results show that DRaCALA is a rapid method for determining affinity and kinetics of protein-DNA interactions.

### Protein Binding of Whole Plasmid Ligands is Detected Specifically by DRaCALA

The mobility of both nucleotides and double stranded oligonucleotides on nitrocellulose suggests that molecular weight is not a critical limiting factor for what types of molecules can be used as the mobile, detectable ligand. The size limit of DNA ligands in DRaCALA was tested by cloning the same ICAP binding site and mutant sites onto a 3.5 kb pVL-Blunt plasmid, and using the entire linearized vector as a ligand. Each of the linearized plasmids were labeled with P³² and shown to be mobile in DRaCALA (plasmids listed in Table 11). Plasmids (50 pM) with ICAP sites bound 100 nM CRP. In contrast, plasmids with 8:GC bound weakly and 10:GC or 8,10:GC sites did not bind at all (FIG. 16A).

Binding of a single ICAP insert on a plasmid probe (50 pM) to 100 nM CRP was next subjected to competition. Competitors in this case were made by PCR amplification of a 600 bp region of the plasmids containing wild type and mutant ICAP sites. The wild type PCR competitor partially inhibited radiolabeled plasmid binding to CRP at 10-fold excess of the radiolabeled ligand and fully competed at 1000-fold excess (FIG. 16B). PCR products containing 8:GC, 10:GC, or 8,10:GC did not compete away binding even at 1000-fold excess concentration. Detected binding of CRP to whole plasmid probes is therefore also site-specific in DRaCALA. These results show that the critical parameter for detection of protein-DNA interaction by DRaCALA is the mobility of the ligand on the solid support and not the molecular weight of the ligand.

### Affinity and Kinetics Determined for Whole Plasmid Ligand

Whole plasmids can also be used in affinity and kinetic studies. With 200 μM cAMP, the observed K_{d} of CRP and a plasmid with a single ICAP site was 7.98±0.82×10⁻¹⁰ M (S.D.) (FIG. 17A). Without cAMP the K_{d} was 2.7±0.46×10⁻⁶ M (S.D.). At only 200 nM cAMP, binding occurred with K_{d} value of 2.8±0.25×10⁻⁸ M (S.D.). These values are similar to those obtained for the labeled oligonucleotides and those from previous studies (Table 9). The off-rate for the plasmid was observed at k_{off}=4.8±0.17×10⁻⁴ s⁻¹, corresponding to a half-life of 23.9 minutes (FIG. 17B). The calculated on-rate for the plasmid was kₒₙ=6.1×10⁵ M⁻¹s⁻¹, which is almost a log lower than that of the annealed oligonucleotides, likely due to the large excess of nonspecific DNA in the plasmid probe. Affinity and kinetics can thus also be measured for sites contained on a plasmid.

### Use of DNA as a Carrier/Label Molecule

Because such large pieces of DNA can be used in DRaCALA without altering specificity, we hypothesized that DNA could be used as a label and carrier for molecules that are not ordinarily mobile in DRaCALA and/or not easily labeled. Because ligand mobility and ligand detection are the only requirements for the mobile binding partner, DNA-conjugation could potentially make any molecule adaptable for use as a DRaCALA probe. A DNA component to the probe allows for easy labeling with ³²P. Many small, soluble molecules are not mobile in DRaCALA suggesting that fluorescently labeled low molecular weight ligand are not suitable for DRaCALA technique using nitrocellulose as a solid support (FIG. 20). However, addition of DNA to immobile ethidium bromide conferred mobility to the interacting dye (FIG. 20) suggesting that conjugation to DNA can overcome the immobility of some dye molecules. DNA can also be covalently linked to molecules through bioconjugate PCR with modified primers. This technique was tested using the biotin-streptavidin system. PCR products including the binding sites of the 3×ICAP plasmid and 3×8,10:GC plasmid were generated with a 5′-biotinylated primer and labelled with ³²P on the free 5′ end. These bioconjugate probes were tested with DRaCALA for binding to CRP, streptavidin and MBP. The wild-type probe with no biotin bound CRP but not streptavidin or MBP (FIG. 18A). The biotinylated wild-type probe bound both CRP and streptavidin but not MBP. The 8,10:GC probe without biotin bound none of the proteins, whereas the biotinylated version bound only streptavidin.

The affinity of the biotinylated ICAP probe was determined using DRaCALA by diluting streptavidin (FIG. 18B). The affinity was limited by the concentration of the probe, which could not be diluted below tens of pM without loss of signal. The limit of DRaCALA detecting binding seems to be therefore the limit of detection of the probe. The IC₅₀ of free biotin was determined by competing against the probe with different concentrations of free biotin (FIG. 18C). Here the IC₅₀ of 33 nM is approximately enough to occupy the 4 sites of the 10 nM streptavidin. The observed affinity is lower than the previous published values for free biotin probably because the biotin molecule was conjugated to DNA. We were also able to measure the off-rate of the conjugated biotin by observing the exchange with excess free biotin (FIG. 21). The exchange occurred in two steps, with an initial rapid off-rate and then a second slower rate corresponding to a half-life of 112 hours and exchange-rate of k_{off}=1.7×10⁻⁶ s⁻¹. The two-step rate has been previously reported in a study of avidin and unconjugated biotin and is likely due to the tetramer protein having different affinities for biotin depending on the number of occupied sites. These results demonstrate that PCR conjugation can be used to link a molecule/ligand of interest to DNA, which allows facile ³²P-labeling and can confer mobility (in DRaCALA), allowing rapid determination of affinity and kinetics of the protein-ligand interaction.

Riboswitch Binding cdiGMP

We have shown that protein interaction with DNA can be detected by DRaCALA. We wondered if the principle of DRaCALA would also apply to ribonucleic acids. In particular, can the DRaCALA technology be used to detect the interaction of riboswitches with their small molecule ligands. One example of such an interaction that has been of recent interest is the cdiGMP responsive Vc2 riboswitch identified in bacteria. To study such an interaction with DRaCALA, one of the binding partners must be immobilized. We achieved this through biotinylation of Vc2* riboswitch RNA (with a modified tetraloop and shortened 5′ and 3′ ends compared to the original Vc2) at the 3′ end by periodate cleavage of the terminal ribose and reductive amination to conjugate the biotin moeity. The biotinylated riboswitch was sequestered by streptavidin, allowing the nucleic acid to take the place of protein as the immobile partner in the binding assay. Vc2* was tested directly for sequestration of cdiGMP and also biotinylated and tested for binding to cdiGMP in the presence or absence of streptavidin. The 4 nM radiolabelled cdiGMP was mobile alone and in the presence of the Vc2* or biotinylated Vc2* RNA (FIG. 19A, lanes 1-3). This suggests that RNA, like DNA, is mobile in this system, and therefore could be used as a labeled probe as well. Streptavidin did not sequester radiolabeled cdiGMP alone or with Vc2* RNA, so there is no detectable interaction between streptavidin and Vc2* RNA (lanes 4-5). Biotinylated Vc2* RNA and bound cdiGMP was immobilized by streptavidin as expected (lane 6). The affinity of Vc2* for cdiGMP was tested using both DRaCALA and an electrophoretic mobility shift assay (EMSA or gel shift). These measurements were made in a Vc2 binding buffer (10 mM sodium cacodylate, 10 mM MgCl₂, 10 mM KCl) by heating the binding reaction to 70° C. for 3 minutes, slowly cooling to room temperature, and then incubating at room temperature for 48 hours. Remarkably similar results were obtained using DRaCALA and gel shift (FIG. 19B). The affinity of the Vc2* RNA for cdiGMP was observed to be K_{d}=7.8±1.9×10⁻⁹ M with DRaCALA and K_{d}=9.8±1.6×10⁻⁹ M with EMSA. These results show that DRaCALA works as well as EMSA for studying the molecular interactions of riboswitches. This strategy can be adapted to study interactions between the biotinylated nucleic acids and a mobile ligand (another nucleic acid or nucleotide).

### Example 3

The following materials and methods were used to demonstrate various embodiments of the invention which pertain to determining protein-ligand binding, particularly for detectably labeled non-nucleic acid ligands, certain specific but non-limiting demonstrations of which are shown in FIGS. 1-5.

### Protein Purification

E. coli strain BL21(DE3) harboring a modified pET19 expression vector (pVL847) expressing an N-terminal histidine-MBP-Alg44 were induced for 6 hours at 30° C. with 1 mM IPTG. Induced bacteria were collected by centrifugation and resuspended in His Buffer A (10 mM Tris, 100 mM NaCl and 25 mM imidazole, pH8.0) and frozen at −80° C. until purification. After addition of DNase, lysozyme and PMSF (1 mM final concentration), thawed bacteria were lysed by sonication. Insoluble material was removed by centrifugation and the His-fusion protein was purified from the clarified whole cell lysate by separation over a Ni-NTA column. Additional information on protein purification is provided below.

### Differential Radial Capillary Action of Ligand Assay

Protein or whole cell lysates in 1× cdiGMP binding buffer (20 μl) was mixed with 4 nM of radiolabeled nucleotide and allowed to incubate for 10 minutes at room temperature. Radiolabeled nucleotide was competed away by cold nucleotides in concentrations and for times indicated. Purified proteins were tested in technical replicates. Whole cell lysates in FIG. 4 and FIG. 12 were tested in biological triplicates. Whole cell lysates in FIG. 5 were tested in technical replicates. These mixtures were pipetted (2.5-5 μl) onto dry, untreated nitrocellulose (GE Healthcare) in triplicate and allowed to dry completely before quantification. An FLA7100 Fujifilm Life Science Phosphorimager was used to detect luminescence following a 5-minute exposure of blotted nitrocellulose to phosphorimager film. Data was quantified using Fujifilm Multi Gauge software v3.0.

### Whole Cell Lysate Preparation

BL21(DE3) cells expressing pVL847 (MBP), pVL882 (MBP-Alg44) or Alg44 point mutations were grown in LB at 30° C., and induced for overexpression with 100 μM IPTG. All Pseudomonas strains from FIG. 5A and Table 6 were grown for 16 hours in LB broth at 37° C. with 200 rpm shaking. Growth conditions of all samples in FIG. 5B and Table 7 are as further described in this Example.

The following materials and methods were used to demonstrate various embodiments of the invention, particularly for certain specific but non-limiting demonstrations of the invention which are shown in FIGS. 6-12.

Detailed Protein Purification.

E. coli strain BL21(DE3) harboring a modified pET19 expression vector (pVL847) expressing an N-terminal histidine-MBP-Alg44 was induced for 6 h at 30° C. with 1 mM isopropyl-β-D-thiogalactopyranoside. Induced bacteria were collected by centrifugation and resuspended in His Buffer A [10 mM Tris, 100 mM NaCl, and 25 mM imidazole (pH8.0)] and frozen at −80° C. until purification. After addition of DNase, lysozyme, and PMSF (1-mM final concentration), thawed bacteria were lysed by sonication. Insoluble material was removed by centrifugation, and the His-fusion protein was purified from the clarified whole-cell lysate by separation over a Ni-NTA column.

His Affinity Purification.

Clarified whole-cell lysates were loaded onto a 10-mL column containing Ni-NTA resin. The Ni-NTA column was washed with 120 mL of His Buffer A to remove non-specifically bound proteins. Elution of the His-tagged protein was accomplished by linearly increasing the imidazole concentration from 25 to 250 mM over 30 mL. Eluted proteins were pooled and dialyzed twice against 40 volumes of 100 mM NaCl and 10 mM Tris (pH 8.0).

Anion Exchange Purification.

The dialyzed eluent from Ni-NTA was loaded onto a 5-mL Q-Sepharose anion exchange column, followed by a wash with 120 mL of 10 mM Tris (pH 8.0) and 100 mM NaCl. Proteins were eluted by linearly increasing the concentration of NaCl from 100 to 500 mM over an 80-mL volume. Eluent fractions containing the protein of interest were pooled, dialyzed twice against 40 volumes of 100 mM NaCl and 10 mM Tris (pH 8.0) supplemented with 25% glycerol, and frozen at −80° C. until thawed for use. Protein concentration was determined by absorbance 280 nm and calculated using a predicted extinction coefficient as determined by the ProtParam program at the ExPASy Web site (expasy.org/tools/protparam.html).

Whole-Cell Lysate Preparation.

Samples from FIG. 5B and Table 8, with the exception of those listed below, were grown in LB broth at 37° C. with 200 rpm shaking. Samples 75, 90, and 91 were grown on YPD plates at 30° C.; sample 74 was grown on a TSB plate in an anaerobic chamber at 37° C.; samples 16, 58, 67, 70, and 79 were grown in TSB broth at 37° C.; samples 83, 84, 85, and 86 were grown in THB broth at 37° C. samples 20, 42, 46, 50, and 89 are tissue samples; sample 51 was grown in Marine Media at 30° C. with shaking; sample 37 was grown in LB broth supplemented with 1 M NaCl; samples 5, 6, 7, 49, and 63 were grown in LB broth at 30° C.; samples 93, 94, 95, and 96 were grown in DMEM F12 from Gibco (catalog no. 10565) supplemented with 10% FBS, 1% penicillin/streptomycin, and 1% glutamine; sample 92 was grown in a 50/50 mixture of Sigma Media 199 (catalog no. M7528) and Sigma Schneider's Complete Media (catalog no. S0146) supplemented with 10% FBS, 1% glutamine, and 1% penicillin/streptomycin; sample 3G_{—}11 Mycobacterium smegmatis (strain mc2 155) was grown in modified 7H9 medium (Difco) as previously described (1); and samples 3H_{—}10 and 3A_{—}11 Neisseria gonorrheae and 3B_{—}11 Neisseria sicca were grown in phosphate-buffered gonococcal medium (Difco) supplemented with 20 mM D-glucose and growth supplements in broth with the addition of 0.042% NaHCO₃ in a CO₂ incubator at 37° C. (2). All bacterial samples were collected by centrifugation, and all tissues were collected by dissection and resuspended in 1/10th volume of 1× cdiGMP binding buffer [100 mM KCl, 5 mM MgCl₂, 100 mM Tris (pH 8.0), and 100 [μM PMSF]. Bacterial samples were also supplemented with lysozyme and DNase. Cells were lysed by two 10-s sonication pulses with 1 min recovery on ice or by bead beating using the Q-Bio lysis system. Extracts were flash-frozen in liquid nitrogen and stored at −80° C. After thawing, 10 μL of whole-cell lysates was incubated with 8 nM ³²P-cdiGMP for 45 s before spotting 2-μL drops on nitrocellulose using an eight-channel pipette.

TABLE 1
Average and SD of lₜₒₜₐₗ for the triplicate DRaCALA
spots depicted in FIG. 2A

| | lₜₒₜₐₗ | cAMP | ATP | cdiGMP |
|---|---|---|---|---|
| MBP | Average | 1,533 | 40,980 | 110,179 |
| | SD | 132 | 1,159 | 2,138 |
| CRP | Average | 2,479 | 38,352 | 112,918 |
| | SD | 177 | 5,277 | 4,038 |
| NtrB | Average | 2,143 | 23,465 | 99,336 |
| | SD | 73 | 1,836 | 3,145 |
| Alg44 | Average | 2,291 | 40,277 | 116,184 |
| | SD | 217 | 2,307 | 1,458 |

TABLE 2
Average and SD of lₜₒₜₐₗ for the triplicate DRaCALA
spots depicted in FIG. 2B

| | lₜₒₜₐₗ | | |
|---|---|---|---|
| Competitor, | First | | Third |
| 1 mM | triplicate | Second triplicate | triplicate |
| No competitor | 47,293 | 46,379 | 43,335 |
| cdiGMP | 30,609 | 31,213 | 34,001 |
| GTP | 42,359 | 45,655 | 41,391 |
| GDP | 42,526 | 40,561 | 42,042 |
| GMP | 40,367 | 48,893 | 46,280 |
| cGMP | 39,354 | 40,539 | 43,532 |
| ATP | 39,782 | 49,712 | 37,376 |
| CTP | 42,686 | 42,958 | 33,382 |
| UTP | 41,575 | 43,598 | 35,762 |
| Average | 40,728 | 43,279 | 39,678 |
| SD | 4,456 | 5,577 | 4,648 |

TABLE 3
Average and SD of lₜₒₜₐₗ for the triplicate DRaCALA
spots depicted in FIG. 3A

| [Alg44_{PilZ}], μM | lₜₒₜₐₗ |
|---|---|
| 100.000 | 77,021 |
| 50.000 | 82,563 |
| 25.000 | 86,246 |
| 12.500 | 86,809 |
| 6.250 | 94,695 |
| 3.125 | 87,742 |
| 1.563 | 96,559 |
| 0.781 | 86,216 |
| 0.391 | 93,135 |
| 0.195 | 83,710 |
| 0.098 | 93,767 |
| 0.049 | 87,169 |
| 0.000 | 77,804 |
| Average | 86,664 |
| SD | 5,704 |

TABLE 4
Average and SD of lₜₒₜₐₗ for the triplicate DRaCALA
spots depicted in FIG. 3C

| | First | | Third |
|---|---|---|---|
| Time(s) | triplicate | Second triplicate | triplicate |
| 0 | 109,607 | 82,354 | 83,547 |
| 10 | 83,366 | 72,748 | 70,773 |
| 15 | 84,462 | 70,645 | 73,335 |
| 20 | 82,922 | 69,334 | 70,831 |
| 30 | 80,647 | 68,939 | 68,335 |
| 45 | 81,803 | 68,864 | 67,199 |
| 60 | 81,468 | 66,620 | 66,282 |
| 90 | 83,626 | 69,013 | 66,519 |
| 120 | 77,729 | 66,088 | 65,528 |
| 180 | 78,442 | 65,317 | 63,472 |
| Average | 84,407 | 69,992 | 69,582 |
| SD | 9,121 | 4,866 | 5,709 |

TABLE 5
Average and SD of lₜₒₜₐₗ for the triplicate DRaCALA spots of
whole-celll lysates depicted in FIG. 4A
BL21(DE3) whole-cell lysates

| | | lₜₒₜₐₗ | | |
|---|---|---|---|---|
| | Competitor, | First | | Third |
| Alg44_{PilZ} | 1 mM | triplicate | Second triplicate | triplicate |
| WT | cdiGMP | 15,895 | 18,914 | 16,999 |
| | GTP | 17,215 | 25,180 | 20,686 |
| R21A | cdiGMP | 18,611 | 22,763 | 19,111 |
| | GTP | 23,957 | 25,116 | 23,905 |
| S46A | cdiGMP | 17,251 | 23,318 | 20,944 |
| | GTP | 15,022 | 24,176 | 21,991 |
| D44A | cdiGMP | 14,558 | 20,624 | 19,909 |
| | GTP | 16,684 | 22,550 | 19,379 |
| R17A, R21A | cdiGMP | 20,636 | 22,338 | 21,226 |
| | GTP | 21,244 | 23,423 | 18,439 |
| Average | | 18,107 | 22,840 | 20,259 |
| SD | | 3,005 | 1,934 | 1,945 |

TABLE 6
Average and SD of lₜₒₜₐₗ for the triplicate
DRaCALA spots of purified proteins depicted in FIG. 4A
Purified proteins

| Alg44_{PilZ} | Competitor, 1 mM | lₜₒₜₐₗ |
|---|---|---|
| WT | cdiGMP | 14,315 |
| | GTP | 16,387 |
| R21A | cdiGMP | 13,676 |
| | GTP | 14,449 |
| S46A | cdiGMP | 13,636 |
| | GTP | 13,921 |
| D44A | cdiGMP | 15,080 |
| | GTP | 15,897 |
| R17A, R21A | cdiGMP | 14,553 |
| | GTP | 14,840 |
| Average | | 14,680 |
| SD | | 909 |

TABLE 7
DRaCALA analysis of cdiGMP binding by whole-cell lysates of
P. aeruginosa strains

| Plate | | | | | | |
|---|---|---|---|---|---|---|
| well | Strain name | Source | B_{G}^{†} | B_{C}^{‡} | B_{Sp}^{§} | A₂₈₀^{¶} |
| 1_A1 | PA15 | UTI | 0.213 | 0.169 | 1.26 | 51.3 |
| 1_B1 | PA14 | UTI | 0.151 | 0.148 | 1.02 | 19.4 |
| 1_C1 | PA13 | UTI | 0.241 | 0.166 | 1.45 | 52.8 |
| 1_D1 | PA8 | UTI | 0.232 | 0.183 | 1.27 | 54.2 |
| 1_E1 | CPs 433 | CF | 0.175 | 0.165 | 1.06 | 45.3 |
| 1_F1 | CPs 433 | CF | 0.228 | 0.162 | 1.41 | 38.8 |
| 1_G1 | CPs 231 | CF | 0.285 | 0.176 | 1.62 | 53.1 |
| 1_H1 | CPs 204 | CF | 0.259 | 0.160 | 1.62 | 50.0 |
| 1_A2 | PAK | Hospital/ | 0.295 | 0.182 | 1.62 | 55.4 |
| | | laboratory | | | | |
| 1_B2 | IT-01 | ATCC | 0.248 | 0.198 | 1.25 | 59.2 |
| 1_C2 | IT-02 | ATCC | 0.193 | 0.163 | 1.18 | 43.3 |
| 1_D2 | IT-03 | ATCC | 0.310 | 0.185 | 1.68 | 60.6 |
| 1_E2 | IT-04 | ATCC | 0.289 | 0.171 | 1.69 | 50.1 |
| 1_F2 | IT-05 | ATCC | 0.271 | 0.184 | 1.47 | 53.7 |
| 1_G2 | IT-06 | ATCC | 0.273 | 0.171 | 1.60 | 41.3 |
| 1_H2 | IT-07 | ATCC | 0.288 | 0.169 | 1.70 | 43.6 |
| 1_A3 | IT-08 | ATCC | 0.273 | 0.171 | 1.60 | 54.1 |
| 1-B3 | IT-09 | ATCC | 0.208 | 0.161 | 1.30 | 38.1 |
| 1_C3 | IT-010 | ATCC | 0.263 | 0.172 | 1.53 | 51.4 |
| 1_D3 | IT-011 | ATCC | 0.246 | 0.165 | 1.49 | 47.1 |
| 1_E3 | IT-013 | ATCC | 0.267 | 0.164 | 1.63 | 44.4 |
| 1_F3 | IT-015 | ATCC | 0.280 | 0.169 | 1.65 | 50.4 |
| 1_G3 | IT-016 | ATCC | 0.257 | 0.167 | 1.54 | 54.1 |
| 1_H3 | IT-017 | ATCC | 0.246 | 0.180 | 1.37 | 59.0 |
| 1_A4 | IT-018 | ATCC | 0.256 | 0.174 | 1.47 | 40.3 |
| 1_B4 | IT-019 | ATCC | 0.188 | 0.174 | 1.08 | 35.5 |
| 1_C4 | IT-020 | ATCC | 0.250 | 0.177 | 1.41 | 49.2 |
| 1_D4 | A 2 A | CF | 0.242 | 0.165 | 1.46 | 50.0 |
| 1_E4 | A 2 B | CF | 0.206 | 0.157 | 1.31 | 37.4 |
| 1_F4 | A 3 | CF | 0.214 | 0.155 | 1.38 | 42.6 |
| 1_G4 | A 7 | CF | 0.266 | 0.171 | 1.55 | 53.5 |
| 1_H4 | A 8 | CF | 0.207 | 0.156 | 1.33 | 35.9 |
| 1_A5 | A 9B | CF | 0.193 | 0.161 | 1.20 | 28.3 |
| 1_B5 | A 10A | CF | 0.235 | 0.174 | 1.35 | 34.1 |
| 1_C5 | A 15A | CF | 0.275 | 0.173 | 1.59 | 46.9 |
| 1_D5 | A 15B | CF | 0.251 | 0.173 | 1.45 | 44.5 |
| 1_E5 | SE1 | CF | 0.218 | 0.162 | 1.34 | 55.2 |
| 1_F5 | SE4 | CF | 0.253 | 0.173 | 1.47 | 45.6 |
| 1_G5 | SE5 | CF | 0.215 | 0.178 | 1.21 | 39.9 |
| 1_H5 | SE8 | CF | 0.198 | 0.159 | 1.25 | 37.4 |
| 1_A6 | SE9A | CF | 0.215 | 0.201 | 1.07 | 38.9 |
| 1_B6 | SE10A | CF | 0.226 | 0.182 | 1.24 | 42.9 |
| 1_C6 | SE11 | CF | 0.226 | 0.169 | 1.34 | 44.9 |
| 1_D6 | SE12B | CF | 0.200 | 0.157 | 1.27 | 51.4 |
| 1_E6 | SE13 | CF | 0.217 | 0.174 | 1.25 | 37.5 |
| 1_F6 | SE14 | CF | 0.220 | 0.174 | 1.26 | 44.3 |
| 1_G6 | SE16 | CF | 0.193 | 0.170 | 1.14 | 36.3 |
| 1_H6 | SE17 | CF | 0.201 | 0.164 | 1.23 | 40.3 |
| 1_A7 | SE19 | CF | 0.202 | 0.172 | 1.17 | 19.9 |
| 1_87 | SE21A | CF | 0.204 | 0.168 | 1.21 | 16.5 |
| 1_C7 | SE21C | CF | 0.202 | 0.172 | 1.17 | 18.1 |
| 1_D7 | SE22B | CF | 0.205 | 0.178 | 1.15 | 14.4 |
| 1_E7 | MI3A | CF | 0.240 | 0.171 | 1.41 | 22.2 |
| 1_F7 | MI3B | CF | 0.246 | 0.180 | 1.36 | 20.2 |
| 1_G7 | MI4A | CF | 0.277 | 0.180 | 1.54 | 21.9 |
| 1_H7 | MI4B | CF | 0.272 | 0.184 | 1.48 | 22.7 |
| 1_A8 | MI5A | CF | 0.249 | 0.177 | 1.41 | 58.6 |
| 1_B8 | MI5B | CF | 0.270 | 0.175 | 1.54 | 51.7 |
| 1_C8 | MI6 | CF | 0.269 | 0.181 | 1.49 | 59.7 |
| 1_D8 | MI8 | CF | 0.242 | 0.166 | 1.46 | 48.3 |
| 1_E8 | MI9A | CF | 0.215 | 0.158 | 1.36 | 40.8 |
| 1_F8 | MI9B | CF | 0.218 | 0.171 | 1.27 | 37.5 |
| 1_G8 | MI9C | CF | 0.231 | 0.173 | 1.34 | 37.7 |
| 1_H8 | MI11A | CF | 0.277 | 0.172 | 1.61 | 30.2 |
| 1_A9 | MI11C | CF | 0.225 | 0.176 | 1.28 | 42.1 |
| 1_B9 | 6073 | Corneal | 0.267 | 0.184 | 1.45 | 54.9 |
| 1_C9 | 6206 | Corneal | 0.310 | 0.182 | 1.70 | 56.2 |
| 1_D9 | 6382 | Corneal | 0.301 | 0.183 | 1.64 | 57.0 |
| 1_E9 | 6389 | Corneal | 0.272 | 0.175 | 1.56 | 48.7 |
| 1_F9 | 6452 | Corneal | 0.238 | 0.178 | 1.34 | 42.9 |
| 1_G9 | PAO1 | Wound/laboratory | 0.281 | 0.187 | 1.51 | 53.4 |
| 1_H9 | 696 | ATCC | 0.204 | 0.167 | 1.22 | 32.8 |
| 1_A10 | 762 | ATCC | 0.293 | 0.172 | 1.70 | 60.0 |
| 1_B10 | 769 | ATCC | 0.233 | 0.170 | 1.37 | 53.0 |
| 1_C10 | 27853 | ATCC | 0.257 | 0.169 | 1.52 | 57.4 |
| 1_D10 | 6354 | Corneal | 0.331 | 0.173 | 1.92 | 57.2 |
| 1_E10 | 6487 | Corneal | 0.256 | 0.170 | 1.50 | 42.8 |
| 1_F10 | PAO381 | CF | 0.304 | 0.173 | 1.76 | 45.8 |
| 1_G10 | PAO578I | Mucoid | 0.322 | 0.167 | 1.93 | 48.8 |
| 1_H10 | PAO578II | Mucoid | 0.320 | 0.165 | 1.94 | 52.2 |
| 1_A11 | PAO579 | Mucoid | 0.292 | 0.166 | 1.77 | 43.0 |
| 1_B11 | PA27853 | ATCC | 0.295 | 0.180 | 1.63 | 55.1 |
| 1_C11 | MCW0001 | CF | 0.281 | 0.176 | 1.60 | 49.8 |
| 1_D11 | 725 | CF | 0.294 | 0.172 | 1.71 | 45.8 |
| 1_E11 | 1328 | CF | 0.294 | 0.167 | 1.76 | 42.0 |
| 1_F11 | 1641 | CF | 0.312 | 0.173 | 1.81 | 51.8 |
| 1_G11 | 381 | CF | 0.381 | 0.164 | 2.32 | 37.5 |
| 1_H11 | 5781 | CF | 0.280 | 0.167 | 1.68 | 47.8 |
| 1_A12 | PA14 pMMB- | | 0.605 | 0.173 | 3.51 | 30.5 |
| | RocR | | | | | |
| 1_B12 | PA14 ΔpelD | | 0.501 | 0.186 | 2.70 | 34.9 |
| | pmmB:RocR | | | | | |
| 1_C12 | CF27 | | 0.211 | 0.164 | 1.29 | 32.6 |
| 1_D12 | PA14 pmmB- | | 0.205 | 0.172 | 1.19 | 29.6 |
| | WspR | | | | | |
| 1_E12 | PA14 ΔretS | | 0.219 | 0.171 | 1.28 | 27.0 |
| 1_F12 | PA14 | Hospital/ | 0.228 | 0.180 | 1.27 | 37.9 |
| | | laboratory | | | | |
| 1_G12 | PAO1 | Wound/laboratory | 0.256 | 0.180 | 1.42 | 44.0 |
| 1_H12 | PAK | Hospital/ | 0.295 | 0.185 | 1.59 | 50.9 |
| | | laboratory | | | | |
| 2_A1 | MSH18 | Environmental | 0.341 | 0.200 | 1.71 | 16.5 |
| 2_B1 | MSH12 | Environmental | 0.310 | 0.197 | 1.57 | 20.8 |
| 2_C1 | MSH13 | Environmental | 0.259 | 0.195 | 1.33 | 18.1 |
| 2_D1 | MSH | Environmental | 0.328 | 0.200 | 1.64 | 26.4 |
| 2_E1 | H14 | Hospital | 0.369 | 0.200 | 1.85 | 26.1 |
| 2_F1 | H12 | Hospital | 0.392 | 0.209 | 1.87 | 17.7 |
| 2_G1 | H19 | Hospital | 0.377 | 0.200 | 1.89 | 14.5 |
| 2_H1 | H25 | Hospital | 0.214 | 0.112 | 1.90 | 23.9 |
| 2_A2 | H26 | Hospital | 0.379 | 0.203 | 1.87 | 20.2 |
| 2_B2 | MSH3 | Environmental | 0.349 | 0.189 | 1.85 | 29.0 |
| 2_C2 | H28 | Hospital | 0.307 | 0.194 | 1.59 | 16.0 |
| 2_D2 | H17 | Hospital | 0.292 | 0.191 | 1.53 | 30.3 |
| 2_E2 | H27 | Hospital | 0.329 | 0.195 | 1.69 | 27.9 |
| 2_F2 | MSH1 | Environmental | 0.373 | 0.193 | 1.93 | 28.0 |
| 2_G2 | H15 | Hospital | 0.345 | 0.198 | 1.74 | 30.0 |
| 2_H2 | H21 | Hospital | 0.327 | 0.186 | 1.76 | 22.0 |
| 2_A3 | MSH5 | Environmental | 0.377 | 0.182 | 2.07 | 16.9 |
| 2_B3 | H24 | Hospital | 0.235 | 0.175 | 1.34 | 6.0 |
| 2_C3 | H22 | Hospital | 0.374 | 0.192 | 1.95 | 24.3 |
| 2_D3 | H29 | Hospital | 0.312 | 0.205 | 1.53 | 24.8 |
| 2_E3 | H2 | Hospital | 0.394 | 0.199 | 1.98 | 22.5 |
| 2_F3 | Pa | Hospital | 0.409 | 0.208 | 1.97 | 21.5 |
| 2_G3 | MSH17 | Environmental | 0.353 | 0.200 | 1.76 | 21.3 |
| 2_H3 | MSH12 | Environmental | 0.371 | 0.196 | 1.89 | 19.9 |
| 2_A4 | H1 | Hospital | 0.376 | 0.194 | 1.93 | 21.2 |
| 2_B4 | PB2036 | | 0.411 | 0.200 | 2.06 | 20.9 |
| 2_C4 | WR5 | Hospital | 0.294 | 0.195 | 1.50 | 13.1 |
| 2_D4 | PA103 | | 0.368 | 0.213 | 1.72 | 28.0 |
| 2_E4 | MSH11 | Environmental | 0.281 | 0.181 | 1.56 | 26.9 |
| 2_F4 | MSH16 | Environmental | 0.345 | 0.192 | 1.80 | 15.8 |
| 2_G4 | MSH10 | Environmental | 0.315 | 0.188 | 1.67 | 13.4 |
| 2_H4 | H30 | Hospital | 0.309 | 0.187 | 1.65 | 12.9 |
| 2_A5 | H23 | Hospital | 0.290 | 0.205 | 1.41 | 11.6 |
| 2_B5 | H16 | Hospital | 0.306 | 0.185 | 1.65 | 16.9 |
| 2_C5 | S11 | Soil | 0.310 | 0.208 | 1.49 | 22.4 |
| 2_D5 | Nathan II | | 0.344 | 0.192 | 1.79 | 20.0 |
| 2_E5 | PAK | Hospital/ | 0.397 | 0.192 | 2.06 | 22.1 |
| | | laboratory | | | | |
| 2_F5 | S11 | Soil | 0.298 | 0.205 | 1.45 | 20.4 |
| 2_G5 | Pa | | 0.413 | 0.203 | 2.04 | 24.6 |
| 2_H5 | Pa | | 0.333 | 0.198 | 1.68 | 21.8 |
| 2_A6 | PAK* | EMS mutant | 0.378 | 0.201 | 1.88 | 19.8 |
| 2_B6 | PAO2003 | | 0.230 | 0.192 | 1.20 | 14.5 |
| 2_C6 | PA103 | CF | 0.337 | 0.199 | 1.69 | 21.4 |
| 2_D6 | 8823 | CF | 0.300 | 0.192 | 1.56 | 16.6 |
| 2_E6 | BHE08 | Brazil | 0.282 | 0.188 | 1.50 | 17.9 |
| 2_F6 | BHE07 | Brazil | 0.265 | 0.186 | 1.42 | 19.0 |
| 2_G6 | BHE06 | Brazil | 0.326 | 0.197 | 1.66 | 22.8 |
| 2_H6 | BHE05 | Brazil | 0.342 | 0.203 | 1.69 | 18.9 |
| 2_A7 | BHE04 | Brazil | 0.192 | 0.186 | 1.03 | 17.3 |
| 2_B7 | BHE03 | Brazil | 0.268 | 0.190 | 1.41 | 14.7 |
| 2_C7 | B27 | Brazil | 0.308 | 0.205 | 1.51 | 27.5 |
| 2_D7 | V209(Alg⁺⁾ | CF | 0.278 | 0.196 | 1.42 | 22.6 |
| 2_E7 | V209(Alg⁻⁾ | CF | 0.304 | 0.194 | 1.57 | 22.9 |
| 2_F7 | Isolate | CF | 0.373 | 0.192 | 1.94 | 20.5 |
| 2_G7 | Isolate | CF | 0.383 | 0.188 | 2.03 | 19.0 |
| 2_H7 | CF1 | CF | 0.309 | 0.201 | 1.54 | 18.8 |
| 2_A8 | CF2 | CF | 0.310 | 0.191 | 1.62 | 21.8 |
| 2_B8 | CF3 | CF | 0.231 | 0.188 | 1.23 | 13.9 |
| 2_C8 | CF4 | CF | 0.243 | 0.186 | 1.31 | 19.7 |
| 2_D8 | CF5 | CF | 0.295 | 0.194 | 1.52 | 28.0 |
| 2_E8 | CF6 | CF | 0.327 | 0.197 | 1.66 | 29.5 |
| 2_F8 | CF26 | CF | 0.281 | 0.184 | 1.53 | 6.6 |
| 2_G8 | CF27 | CF | 0.318 | 0.181 | 1.75 | 18.0 |
| 2_H8 | CF28 | CF | 0.503 | 0.188 | 2.68 | 19.5 |
| 2_A9 | CF29 | CF | 0.298 | 0.189 | 1.57 | 20.2 |
| 2_B9 | R37 | CF | 0.376 | 0.183 | 2.06 | 8.0 |
| 2_C9 | R71 | CF | 0.276 | 0.193 | 1.43 | 11.6 |
| 2_D9 | 6077 | Corneal | 0.357 | 0.206 | 1.74 | 18.7 |
| 2_E9 | 6294 | Corneal | 0.368 | 0.199 | 1.861 | 1.1 |
| 2_F9 | 19660 | Corneal | 0.327 | 0.199 | 1.652 | 4.6 |
| 2_G9 | F34842 | UTI | 0.362 | 0.206 | 1.751 | 8.6 |
| 2_H9 | F35896 | UTI | 0.356 | 0.209 | 1.712 | 4.5 |
| 2_A10 | H38036 | UTI | 0.282 | 0.197 | 1.432 | 0.6 |
| 2_B10 | M28497 | UTI | 0.263 | 0.205 | 1.291 | 9.2 |
| 2_C10 | W57761 | UTI | 0.300 | 0.214 | 1.402 | 5.1 |
| 2_D10 | X24509 | UTI | 0.402 | 0.215 | 1.872 | 5.0 |
| 2_E10 | UTI121 | UTI | 0.331 | 0.204 | 1.621 | 9.7 |
| 2_F10 | UTI122 | UTI | 0.339 | 0.200 | 1.691 | 5.5 |
| 2_G10 | UTI123 | UTI | 0.277 | 0.200 | 1.381 | 4.5 |
| 2_H10 | UTI124 | UTI | 0.366 | 0.203 | 1.802 | 5.4 |
| 2_A11 | UTI125 | UTI | 0.307 | 0.193 | 1.591 | 0.8 |
| 2_B11 | UTI126 | UTI | 0.267 | 0.201 | 1.331 | 2.2 |
| 2_C11 | UTI127 | UTI | 0.287 | 0.200 | 1.441 | 9.3 |
| 2_D11 | B312 | Blood | 0.313 | 0.204 | 1.531 | 7.1 |
| 2_E11 | B1460A | Blood | 0.321 | 0.203 | 1.582 | 1.4 |
| 2_F11 | B1874-2 | Blood | 0.335 | 0.203 | 1.651 | 9.9 |
| 2_G11 | CF32 | CF | 0.328 | 0.215 | 1.532 | 1.6 |
| 2_H11 | U130 | UTI | 0.365 | 0.210 | 1.741 | 5.3 |
| 2_A12 | U169 | UTI | 0.255 | 0.190 | 1.342 | 5.5 |
| 2_B12 | U779 | UTI | 0.340 | 0.218 | 1.563 | 6.0 |
| 2_C12 | U2504 | UTI | 0.303 | 0.206 | 1.473 | 5.5 |
| 2_D12 | H21651 | Blood | 0.323 | 0.212 | 1.533 | 3.6 |
| 2_E12 | X13397 | Blood | 0.310 | 0.207 | 1.503 | 9.4 |
| 2_F12 | X16259 | Blood | 0.276 | 0.202 | 1.372 | 6.6 |
| 2_G12 | S29712 | Blood | 0.331 | 0.208 | 1.592 | 4.0 |
| 2_H12 | S35004 | Blood | 0.347 | 0.225 | 1.543 | 5.6 |

| | |
|---|---|
| Average B_{c =} | 0.185 |
| SD B_{c =} | 0.016 |
| 2 * SD B_{c =} | 0.031 |
| B_{Sp} positive cutoff = | 1.17 |

*Strains are classified as indicated. CF, cystic fibrosis isolate; UTI, urinary tract infection isolate; ATCC, American Type Culture Collection.
^{†}³²P-cdiGMP bound during 1 mM GTP competition.
^{‡}³²-cdiGMP bound during 1 mM cdiGMP competition.
^{§}Specific binding of whole-cell lysate (B_{G}/B_{C}).
^{¶}Total protein concentration of whole-cell lysate measured by absorbance at 280 nM by a Thermo Fischer Nanodrop 8000 using 0.2-μM path length.
Absorbance units are reported as if measured with 10-mm path length (actual path length of 0.2 mm).

TABLE 8
DRaCALA analysis of cdiGMP binding by lysates from various organisms or tissues

| Plate | | | Strain/tissue/cell | | | | Predicted | Reference for |
|---|---|---|---|---|---|---|---|---|
| well | Genus | Species | type | B_{G}† | B_{C}‡ | B_{Sp}§ | DGC^{¶} | cdiGMP signaling^{\|\|} |
| 3_A1 | Aeromonas | hydrophila | SJ11R | 0.248 | 0.162 | 1.53* | Yes | None |
| 3_B1 | Salmonella | typhimurium | SL1334 | 0.164 | 0.153 | 1.07 | Yes | (1) |
| 3_C1 | Escherichia | coli | ZK57 | 0.149 | 0.141 | 1.06 | Yes | (1) |
| 3_D1 | Yersinia | enterocolitica | W22703 | 0.161 | 0.147 | 1.10 | Yes | None |
| 3_E1 | Yersinia | enterocolitica | 8081 | 0.181 | 0.155 | 1.17* | Yes | None |
| 3_F1 | Yersinia | pseudotuberculosis | pIB1 | 0.155 | 0.158 | 0.99 | Yes | None |
| 3_G1 | Yersinia | pseudotuberculosis | pYPIII | 0.152 | 0.150 | 1.01 | Yes | None |
| 3_H1 | Escherichia | coli | JM109 | 0.171 | 0.165 | 1.03 | Yes | (1) |
| 3_A2 | Vibrio | cholerae | N16961 | 0.354 | 0.179 | 1.98* | Yes | (2) |
| 3_B2 | Burkholderia | dolosal | HI2914 | 0.301 | 0.166 | 1.82* | Yes | None |
| 3_C2 | Burkholderia | dolosal | AU3960 | 0.278 | 0.178 | 1.56* | Yes | None |
| 3_D2 | Bacillus | subtilis | 3160 | 0.185 | 0.162 | 1.14 | Yes | (3) |
| 3_E2 | Bacillus | subtilis | 168 | 0.189 | 0.157 | 1.20* | Yes | (3) |
| 3_F2 | Bacillus | subtilis | PY79 | 0.206 | 0.152 | 1.36* | Yes | (3) |
| 3_G2 | Actinomyces | naeslundii | MG1 | 0.162 | 0.159 | 1.02 | No | None |
| | | | | | | | sequence | |
| 3_H2 | Staphylococcus | aureus | Newman | 0.148 | 0.141 | 1.05 | Yes | cdiGMP- |
| | | | | | | | | independent (4, 5) |
| 3_A3 | Streptococcus | agalactiae | 2603 | 0.156 | 0.152 | 1.03 | No | None |
| 3_B3 | Pseudomonas | putida | pB2440 | 0.143 | 0.133 | 1.07 | Yes | (6) |
| 3_C3 | Proteus | mirabilis | SC81cM1061 | 0.158 | 0.165 | 0.96 | Yes | None |
| 3_D3 | Caenorhabditis | elegans | | 0.151 | 0.157 | 0.96 | No | None |
| 3_E3 | Pseudomonas | stutzeri | K2186 | 0.244 | 0.150 | 1.62* | Yes | None |
| 3_F3 | Pseudomonas | stutzeri | K1412 | 0.224 | 0.158 | 1.42* | Yes | None |
| | | | M1035 | | | | | |
| 3_G3 | Pseudomonas | stutzeri | K79 | 0.287 | 0.155 | 1.85* | Yes | None |
| 3_H3 | Pseudomonas | fluorescens | K2122 | 0.277 | 0.159 | 1.74* | Yes | (6) |
| 3_A4 | Stenotrophomonas | maltophilia | K2227 | 0.279 | 0.167 | 1.67* | Yes | None |
| 3_B4 | Brevundimonas | vesicularis | K136 | 0.298 | 0.175 | 1.71* | No | None |
| | | | | | | | sequence | |
| 3_C4 | Providencia | stuartii | SC145 | 0.156 | 0.170 | 0.92 | Yes | None |
| | | | M1062 | | | | | |
| 3_D4 | Pseudomonas | fluorescens | K2017 | 0.207 | 0.169 | 1.23* | Yes | (6) |
| | | | M1088 | | | | | |
| 3_E4 | Burkholderia | cenocepacia | K2313 | 0.152 | 0.163 | 0.93 | Yes | None |
| 3_F4 | Moraxella | osloensis | K1980 | 0.144 | 0.145 | 0.99 | No | None |
| | | | | | | | sequence | |
| 3_G4 | Pseudomonas | fluorescens | E-38 | 0.185 | 0.144 | 1.29* | Yes | (6) |
| 3_H4 | Proteus | mirabilis | H-62 | 0.156 | 0.163 | 0.95 | Yes | None |
| 3_A5 | Proteus | vulgaris | | 0.158 | 0.168 | 0.94 | No | None |
| | | | | | | | sequence | |
| 3_B5 | Pseudomonas | alcaligenes | D13 | 0.313 | 0.174 | 1.80* | No | None |
| | | | | | | | sequence | |
| 3_C5 | Delftia | acidovorans | D12 | 0.324 | 0.177 | 1.83* | Yes | None |
| 3_D5 | Comamona | testosteronis | D14 | 0.279 | 0.164 | 1.70* | Yes | None |
| 3_E5 | Pseudomonas | mendocina | D57 | 0.134 | 0.141 | 0.95 | Yes | None |
| 3_F5 | Stenotrophomonas | maltophilia | C40 | 0.277 | 0.199 | 1.39* | Yes | None |
| 3_G5 | Pseudomonas | putida | C14 | 0.192 | 0.171 | 1.12 | Yes | (6) |
| 3_H5 | Shewanella | putrefaciens | F17 | 0.300 | 0.165 | 1.82* | Yes | None |
| 3_A6 | Pseudomonas | stutzeri | H24 | 0.192 | 0.157 | 1.22* | Yes | None |
| 3_B6 | Nicotiana | benthamiana | | 0.199 | 0.170 | 1.17* | No | None |
| | | | | | | | sequence | |
| 3_C6 | Burkholderia | cenocepacia | F2 | 0.218 | 0.174 | 1.25* | Yes | None |
| 3_D6 | Burkholderia | cenocepacia | F27 | 0.188 | 0.158 | 1.19* | Yes | None |
| 3_E6 | Pseudomonas | diminuta | | 0.211 | 0.162 | 1.30* | No | None |
| | | | | | | | sequence | |
| 3_F6 | Mus | musculus | Brain | 0.315 | 0.243 | 1.29* | No | (7) |
| 3_G6 | Vibrio | cholerae | IRA J13 | 0.328 | 0.169 | 1.94* | Yes | (2) |
| 3_H6 | Klebsiella | pneumoniae | W63917 | 0.160 | 0.155 | 1.03 | Yes | (8) |
| 3_A7 | Sinorhizobium | meliloti | Rm1021 | 0.164 | 0.162 | 1.02 | Yes | None |
| 3_B7 | Mus | musculus | RAW | 0.151 | 0.169 | 0.89 | No | (7) |
| | | | cells | | | | | |
| 3_C7 | Vibrio | harveyi | MM32 | 0.207 | 0.162 | 1.28* | Yes | None |
| 3_D7 | Salmonella | typhimurium | | 0.160 | 0.163 | 0.98 | Yes | (1) |
| 3_E7 | Escherichia | coli | | 0.256 | 0.173 | 1.48* | Yes | (1) |
| 3_F7 | Citrobacter | freundii | | 0.154 | 0.156 | 0.99 | No | None |
| | | | | | | | sequence | |
| 3_G7 | Serratia | marcescens | | 0.204 | 0.170 | 1.20* | No | None |
| | | | | | | | sequence | |
| 3_H7 | Hafnia | alvei | | 0.172 | 0.152 | 1.13 | No | None |
| | | | | | | | sequence | |
| 3_A8 | Micrococcus | luteus | | 0.136 | 0.140 | 0.98 | No | None |
| 3_B8 | Staphylococcus | epidermidis | | 0.145 | 0.155 | 0.94 | Yes | cdiGMP- |
| | | | | | | | | independent (4, 5) |
| 3_C8 | Enterobacter | aerogenes | | 0.169 | 0.152 | 1.11 | No | None |
| | | | | | | | sequence | |
| 3_D8 | Bacillus | megaterium | | 0.164 | 0.147 | 1.12 | Yes | None |
| 3_E8 | Pseudomonas | putida | NCIMB | 0.155 | 0.157 | 0.99 | Yes | (6) |
| 3_F8 | Ochrobactrum | anthropi | NCIMB | 0.222 | 0.168 | 1.32* | Yes | None |
| | | | 8686 | | | | | |
| 3_G8 | Moraxella | catarrhalis | | 0.155 | 0.161 | 0.96 | No | None |
| 3_HB | Acinetobacter | spp. | MD4 | 0.157 | 0.158 | 0.99 | Yes | None |
| 3_A9 | Moraxella | spp. | B88 | 0.154 | 0.149 | 1.03 | No | None |
| 3_B9 | Lactococcus | Lactis | | 0.243 | 0.166 | 1.46* | Yes | None |
| 3_C9 | Staphylococcus | aureus | | 0.197 | 0.162 | 1.21* | Yes | cdiGMP- |
| | | | | | | | | independent (4, 5) |
| 3_D9 | Alcaligenes | faecalis | | 0.172 | 0.164 | 1.05 | No | None |
| | | | | | | | sequence | |
| 3_E9 | Corynebacterium | xerosis | | 0.145 | 0.152 | 0.95 | No | None |
| | | | | | | | sequence | |
| 3_F9 | Staphylococcus | sciuri | | 0.148 | 0.154 | 0.96 | No | None |
| | | | | | | | sequence | |
| 3_G9 | Proteus | mirabilis | | 0.168 | 0.176 | 0.96 | Yes | None |
| 3_H9 | Enterococcus | durans | | 0.155 | 0.154 | 1.01 | No | None |
| | | | | | | | sequence | |
| 3_A10 | Marinococcus | halophilus | | 0.169 | 0.147 | 1.14 | No | None |
| | | | | | | | sequence | |
| 3_B10 | Clostridium | sporogenes | | 0.171 | 0.195 | 0.87 | Yes | None |
| 3_C10 | Saccharomyces | cerevisiae | | 0.160 | 0.159 | 1.01 | No | None |
| 3_D10 | Providencia | stuartii | | 0.157 | 0.166 | 0.95 | Yes | None |
| 3_E10 | Bacillus | cereus | | 0.161 | 0.152 | 1.06 | Yes | (9) |
| 3_F10 | Enterococcus | faecalis | | 0.175 | 0.160 | 1.10 | No | None |
| 3_G10 | Staphylococcus | aureus | MRSA | 0.152 | 0.156 | 0.97 | Yes | cdiGMP- |
| | | | | | | | | independent (4, 5) |
| 3_H10 | Neisseria | gonorrheae | MS11 | 0.151 | 0.150 | 1.01 | No | None |
| 3_A11 | Neisseria | gonorrheae | F11090 | 0.149 | 0.155 | 0.96 | No | None |
| 3_B11 | Neisseria | sicca | | 0.173 | 0.158 | 1.09 | No | None |
| 3_C11 | Streptococcus | pyogenes | GA40634 | 0.145 | 0.146 | 1.00 | Yes | None |
| 3_D11 | Streptococcus | pyogenes | NZ131 | 0.160 | 0.144 | 1.12 | Yes | None |
| 3_E11 | Streptococcus | pyogenes | 5448- | 0.151 | 0.152 | 0.99 | Yes | None |
| | | | AN | | | | | |
| 3_F11 | Streptococcus | pyogenes | GA19681 | 0.153 | 0.149 | 1.03 | Yes | None |
| 3_G11 | Mycobacterium | smegmatis | | 0.156 | 0.156 | 1.00 | Yes | (10) |
| 3_H11 | Aspergillus | niger | | 0.156 | 0.157 | 0.99 | No | None |
| 3_A12 | Mus | musculus | Heart | 0.213 | 0.196 | 1.09 | No | (7) |
| 3_B12 | Saccaromyces | cerevisiae | cry1/cry2 | 0.168 | 0.170 | 0.99 | No | None |
| 3_C12 | Saccaromyces | cerevisiae | AH109 | 0.161 | 0.158 | 1.02 | No | None |
| 3_D12 | Leishmania | major | | 0.154 | 0.170 | 0.91 | No | None |
| 3_E12 | Homo | sapiens | U397 | 0.181 | 0.271 | 0.67 | No | (11) |
| | | | cells | | | | | |
| 3_F12 | Homo | sapiens | HuH7 | 0.149 | 0.151 | 0.99 | No | (11) |
| | | | cells | | | | | |
| 3_G12 | Cricetulus | griseus | CHO | 0.179 | 0.273 | 0.66 | No | None |
| | | | cells | | | | sequence | |
| 3_H12 | Mus | musculus | Spleen | 0.227 | 0.299 | 0.76 | No | (7) |

IRA; MRSA, methicillin-resistant Staphylococcus aureus; NCIMB; RAW.
^{†}³²P-cdiGMP bound in the presence of the nonspecific competitor GTP at 1 mM.
^{‡}³²P-cdiGMP bound in the presence of the specific competitor cdiGMP at 1 mM.
^{§}Specific binding of whole-cell lysate (B_{G}/B_{C}). Organisms with values above the 1.17 cutoff are indicated by asterisks (*).
^{¶}Genomes that encode DGC were identified on Oct. 7, 2010, by a search at www.ncbi.nlm.nih.gov/protein using a search term consisting of the genus and species of each organism, along with “DGC”, “GGDEF”, or “diguanylate”. Organisms positive for DGC are indicated by “Yes.” Organisms negative for DGC are indicated by “No.” Those without a sequenced genome are indicated by “No sequence.”
^{||}References for organisms using cdiGMP signaling were identified by a PubMed search using a search term consisting of the genus and species of each organism and “cyclic-di-GMP” on Oct. 7, 2010. The earliest reference reporting cdiGMP signaling in each species is shown. Organisms for which no citations were available are indicated by “None”. “cdiGMP independent” is noted for those strains that have a protein with a DGC domain and observed regulation that is independent of cdiGMP nucleotide.
(1). Simm R, MorrM, Kaker A, Mimtz M, Römling U (2004) GGDEF and EAL domains inversely regulate cyclic di-GMP levels and transition from sessility to motility. Mol Microbiol 53: 1123-1134.
(2). Tischler AD, Camilli A (2004) Cyclic diguanylate (c-di-GMP regulates Vibrio cholerae biofilm formation. Mol Microbiol 53: 857-869.
(3). Minasov G, et al. (2009) Crystal structures of Ykul and its complex with second messenger cyclic Di-GMP suggest catalytic mechanism of phosphodiester bond cleavage by EAL domains. J Biol Chem 284: 13174-13184.
(4). Holland LM, et al. (2008) A staphylococcal GGDEF domain protein regulates biofilm formation independently of cyclic dimeric GMP. J Bacteriol 190: 5178-5189.
(5). Shang F, et al. (2009) The Staphylococcus aureus GGDEF domain-containing protein, GdpS, influences protein A gene expression in a cyclic diguanylic acid-independent manner. Infect Immun 77: 2849-2856.
(6). Ude S, Arnold DL, Moon CD, Timms-Wilson T, Spiers AJ (2006) Biofilm formation and cellulose expression among diverse environmental Pseudomonas isolates. Environ Microbiol 8: 1997-2011.
(7). Brouillette E, Hyodo M, Hayakawa Y, Karaolis DK, Malouin F (2005) 3′,5′-cyclic diguanylic acid reduces the virulence of biofilm-forming Staphylococcus aureus strains in a mouse model of mastitis infection. Antimicrob Agents Chemother 49: 3109-3113.
(8). Johnson JG, Clegg S (2010) Role of MrkJ, a phosphodiesterase, in type 3 fimbrial expression and biofilm formation in Klebsiella pneumoniae. J Bacteriol 192: 3944-3950.
(9). Sudarsan N, et al. (2008) Riboswitches in eubacteria sense the second messenger cyclic di-GMP. Science 321: 411-413.
(10). Kumar M. Chatterji D (2008) Cyclic di-GMP: A second messenger required for long-term survival, but not for biofilm formation, in Mycobacterium smegmatis. Microbiology 154: 2942-2955, and retraction (2011) 157(Pt 3): 918.
(11). Karaolis DK, et al. (2005) 3′,5′-Cyclic diguanylic acid (c-di-GMP) inhibits basal and growth factor-stimulated human colon cancer cell proliferation. Biochem Biophys Res Commun 329: 40-45.

TABLE 9
Observed affinity of CRP to various ICAP probes from this and
previous studies with indicated amounts of cAMP. All reported
K_{d} values from this study were determined by DRaCALA and
the standard deviation of three trials is reported.

| | | [cAMP] | |
|---|---|---|---|
| Source | Probe | (M) | K_{d} (M) (± S.D.) |
| Gunasekera, 92 (7) | ICAP oligo ³²P | 2 × 10⁻⁴ | 7.0 ± 0.3 × 10⁻¹⁰ |
| Gunasekera, 92 (7) | ICAP oligo ³²P | 0 | >1.0 × 10⁻⁷ |
| Gunasekera, 92 (7) | 10:G-C oligo ³²P | 2 × 10⁻⁴ | >1.0 × 10⁻⁷ |
| Fried, 84 (17) | lac CRP oligo ³²P | 5 × 10⁻⁶ | 8.4 × 10⁻¹⁰ |
| Fried, 84 (17) | lac CRP oligo ³²P | 2 × 10⁻⁷ | 6.3 × 10⁻⁸ |
| This study | ICAP oligo ³²P | 2 × 10⁻⁴ | 5.6 ± 0.46 × 10⁻¹⁰ |
| This study | ICAP oligo ³²P | 2 × 10⁻⁷ | 5.6 ± 0.38 × 10⁻⁸ |
| This study | ICAP oligo ³²P | 0 | 8.4 ± 1.2 × 10⁻⁶ |
| This study | ICAP plasmid ³²P | 2 × 10⁻⁴ | 8.0 ± 0.82 × 10⁻¹⁰ |
| This study | ICAP plasmid ³²P | 2 × 10⁻⁷ | 2.8 ± 0.25 × 10⁻⁸ |
| This study | ICAP plasmid ³²P | 0 | 2.7 ± 0.46 × 10⁻⁶ |
| This study | 10:G-C | 2 × 10⁻⁴ | 1.0 ± 0.14 × 10⁻⁶ |
| | plasmid ³²P | | |
| This study | 10:G-C | 0 | 2.9 ± 0.58 × 10⁻⁶ |
| | plasmid ³²P | | |

### Example 4

The following materials and methods were used to obtain data which demonstrate various embodiments of the invention used for determining nucleic acid ligand/protein binding, particularly as it pertains to certain specific but non-limiting demonstrations of the invention which are shown in FIGS. 13-21.

### Proteins, Nucleic Acids, and Chemicals

The Vc2* DNA template was ordered from Integrated DNA Technologies. Other DNA oligonucleotides, Nucaway size exclusion columns, and Turbo DNase were from Invitrogen. RNase was from Fermentas. RNase inhibitor and enzymes for restriction digests, PCR, and other nucleic acid manipulations were from New England Biolabs. Streptavidin MagneSphere Paramagnetic Particles, Wizard miniprep and PCR Purification kits for DNA purification were from Promega. Biotin hydrazide and streptavidin were from Sigma Aldrich.

CRP was purified according to as described above. Briefly, His-CRP was expressed from pBAD-CRP (a gift from Dr. Sankar Adhya) and purified using a Ni-NTA column. Proteins were dialyzed in 10 mM Tris, pH8.0 and 100 mM NaCl. His-CRP was subsequently purified and concentrated using anion exchange to a concentration of 20 μM, supplemented with 25% glycerol, and stored at −80° C. until thawing for use.

### DNA Oligonucleotides and Plasmid Probes

Reverse complementary oligonucleotides gd126-133 (Table 10) were used to generate probes by labeling 5 pmol of the forward primer with T4 Polynucleotide Kinase (PNK) and 15 pmol/5 mCi of γ-³²P-labelled ATP.

TABLE 10
Primers used in this study. ICAP and mutant ICAP sites are indicated (RC = reverse
complement).

| Name | Content | Use | Sequence (5′ -3′) |
|---|---|---|---|
| gd126 | ICAP | oligonucleotide probe | AGGAGGAATAAATGTGATCTAGATCACATTTTAGAGGAGG |
| | | | (SEQ ID NO: 1) |
| gd127 | ICAP RC | oligonucleotide probe | CCTCCTCTAAAATGTGATCTAGATCACATTTATTCCTCCT |
| | | | (SEQ ID NO: 2) |
| gd128 | ICAP 8: G-C | oligonucleotide probe | AGGAGGAATAAATCTGATCTAGATCAGATTTTAGAGGAGG |
| | | | (SEQ ID NO: 3) |
| gd129 | ICAP 8: G-C RC | oligonucleotide probe | CCTCCTCTAAAATCTGATCTAGATCAGATTTATTCCTCCT |
| | | | (SEQ ID NO: 4) |
| gd130 | ICAP 10: G-C | oligonucleotide probe | AGGAGGAATAAATGTCATCTAGATGACATTTTAGAGGAGG |
| | | | (SEQ ID NO: 5) |
| gd131 | ICAP 10: G-C RC | oligonucleotide probe | CCTCCTCTAAAATGTCATCTAGATGACATTTATTCCTCCT |
| | | | (SEQ ID NO: 6) |
| gd132 | ICAP 8, 10: G-C | oligonucleotide probe | AGGAGGAATAAATCTCATCTAGATGAGATTTTAGAGGAGG |
| | | | (SEQ ID NO: 7) |
| gd133 | ICAP 8, 10: G-C RC | oligonucleotide probe | CCTCCTCTAAAATCTCATCTAGATGAGATTTATTCCTCCT |
| | | | (SEQ ID NO: 8) |
| kr122 | ICAP | clone into plasmid | AATAAATGTGATCTAGATCACATTTTAG (SEQ ID |
| | | | NO: 9) |
| kr123 | ICAP RC | clone into plasmid | CTAAAATGTGATCTAGATCACATTTATT (SEQ ID |
| | | | NO: 10) |
| kr124 | ICAP 8: G-C | clone into plasmid | AATAAATCTGATCTAGATCAGATTTTAG (SEQ ID |
| | | | NO: 11) |
| kr125 | ICAP 8: G-C RC | clone into plasmid | CTAAAATCTGATCTAGATCAGATTTATT (SEQ ID |
| | | | NO: 12) |
| kr126 | ICAP 10: G-C | clone into plasmid | AATAAATGTCATCTAGATGACATTTTAG (SEQ ID |
| | | | NO: 13) |
| kr127 | ICAP 10: G-C RC | clone into plasmid | CTAAAATGTCATCTAGATGACATTTATT (SEQ ID |
| | | | NO: 14) |
| kr128 | ICAP 8, 10: G-C | clone into plasmid | AATAAATCTCATCTAGATGAGATTTTAG (SEQ ID |
| | | | NO: 15) |
| kr129 | ICAP 8, 10: G-C RC | clone into plasmid | CTAAAATCTCATCTAGATGAGATTTATT(SEQ ID |
| | | | NO: 16) |
| v1880 | — | PCR of insert | GACCATGATTACGCCAAGCTA (SEQ ID NO: 17) |
| v1881 | — | PCR of insert | CAGCTTTCATCCCCGATATG (SEQ ID NO: 18) |

Five pmol of the reverse complementary primer were added and the PNK was heat-inactivated during primer annealing in a 95° C. water bath for ten minutes, which was then allowed to cool to room temperature. The annealed product was separated from free ³²P-ATP using a Nucaway column and diluted 1:10 for binding and competitions studies and 1:1000 for affinity and kinetics studies. Plasmids with binding sites were generated by cloning annealed, PNK-treated primers pairs (kr122-129 of Table 10) into Stul-cut pVL-Blunt, and sequencing for verification (Table 11).

TABLE 11
Plasmids used in this study. ICAP and mutant ICAP sites are indicated.

| Name | Parent | Insert |
|---|---|---|
| pVL-Blunt | — | — |
| pGD7 | pVL-Blunt | ICAP x5 |
| pGD8 | pVL-Blunt | ICAP x3 |
| pGD9 | pVL-Blunt | ICAP x1 |
| pGD11 | pVL-Blunt | ICAP 8:G-C x3 |
| pGD12 | pVL-Blunt | ICAP 10:G-C x3 |
| pGD13 | pVL-Blunt | ICAP 8,10:G-C x3 |

Plasmids were 5′ end-labeled by sequential digestion with the single cutter BamHI, dephosphorylation of the 5′ overhang with Calf Intestinal Alkaline Phosphatase, separation from enzymes by a Wizard PCR Purification column, and treatment with PNK in the presence of γ-³²P-labelled ATP. The labeled product was purified by Wizard column and a Nucaway column and diluted 1:10 for affinity and kinetic study. The near 5′ end of these labeled plasmids is about 40 bp from the cloned binding sites. Competitors for plasmid binding were PCR amplified from these plasmids using primers v1880-v1881, which amplify the cloned binding sites and 250 bp flanking on each side (Table 10).

### Differential Radial Capillary Action of Ligand Assay

Protein, ³²P-labeled DNA, and 200 μM cAMP (unless otherwise noted) were mixed in CRP buffer (10 mM Tris pH=7.9, 200 mM NaCl, 0.1 mM DTT, 50 μg/ml BSA) and incubated at room temperature for ten minutes. 5 μl of the mix was spotted on nitrocellulose by first pipetting the liquid out onto the tip of the pipette and then touching the drop to the membrane. Spots were allowed to dry completely (about 20 minutes) before exposing a phosphorimager screen and capturing with a Fujifilm FLA-7000. Photostimulated luminescence (PSL) from the inner spot and total PSL of the spot were quantitated with Fuji Image Gauge software. The fraction bound (F_{b}) was calculated using measurements of the total area (Aₒᵤₜₑᵣ), the area of the inner circle (Aᵢₙₙₑᵣ) the total PSL intensity (Iₜₒₜₐₗ), and the inner intensity (Iᵢₙₙₑᵣ) as follows:

$${F}_{B} = \frac{{I}_{inner} - {A}_{inner} * ( \frac{{I}_{total} - {I}_{inner}}{{A}_{total} - {A}_{inner}} )}{{I}_{total}}$$

### Non-Radioactive Ligands and Detection

Fluorescent dyes were imaged with a GE Typhoon Trio. TNP was detected with electrochemiluminescence excitation at 555 nm emission. FITC was detected with 488 nM excitation and 526 nM emission. Ethidium bromide was imaged under a UV light source. TRITC, Propidium iodide, crystal violet, and coomassie brilliant blue were imaged in visible light.

### Bioconjugate PCR

Biotinylated probes were generated by PCR using 5′-biotinylated primer v1881 for amplification of a ˜600 base pair region of plasmids pGD9 and pGD13 (Table 11). PCR products were extracted from an agarose gel and purified with a Wizard column. These were then γ-³²P-labelled as described for the whole plasmids.

### Preparation and Purification of Vc2* RNA

The Vc2* template sequence including T7 promoter sequence and complimentary T7 promoter sequence 5′-CTA ATA CGA CTC ACT ATA G-3′ (SEQ ID NO:19) were purchased from Integrated DNA Technologies (IDT). Transcription was performed using 1.5 μg of template, 10 μL of 4 mg/ml T7 polymerase per 200 μL of transcription volume, 15 mM total NTP (A/C/G/UTPs), 15 mM MgCl₂ in a transcription buffer of 40 mM Tris-HCl (pH 8.1), 1 mM spermidine, 5 mM dithiothreitol (DTT), 0.01% Trixon X-100, 2 units of RNase inhibitor, 2 unit of inorganic pyrophosphatase. After 3 h, 0.4 units of Turbo DNase were added and incubated for another 15 min. The crude RNA was purified using a 12% denaturing PAGE with 1×TBE buffer. The product band was detected via UV-shadowing the gel, excised and electro-eluted in a Schleicher and Schuell Elutrap eletro-separation system. The purified RNA was precipitated with three volumes of absolute ethanol and 10% volumes of 0.3 M sodium acetate. The RNA pellet was then resuspended in water and dialyzed in a Nestroup Biodialyzer with a 500 MWCO membrane for 24 h against 100 mM potassium phosphate buffer (pH 6.4), 0.5 M KCl, 10 mM EDTA, and then 1 and 0.1 mM EDTA, and finally against two changes of double distilled H₂O water before it was lyophilized.

Biotin Labeling of RNA with Biotin Hydrazide at 3′-End

Seven μL of freshly prepared 0.5 M NaIO₄ was added to Vc2* RNA (210 μg) in 100 μL of water and the solution incubated at room temperature for 1 h. The excess NaIO₄ was removed by filtration, using an Amicon ultra 0.5 mL centrifugal filter with 10K cut-off membrane. The RNA was washed with 3×0.5 mL of water and then recovered by reverse spin. After that, 5 μL of 1M sodium acetate, pH 4.95, and 7 ml of 35 mM biotin hydrazide in DMSO were added to the RNA. Coupling was carried out at 37° C. for 1.5 hr, then 3 μL of 1 M NaCNBH₃ in acetonitrile was added and the reduction was carried out at room temperature for 1 hr. The unused biotin hydrazide and NaCNBH₃ were removed by centrifugal filter as above.

Testing the Biotinylation Efficiency with Magnetic Streptavidin Beads

Four hundred μL of streptavidin MagneSphere Paramagnetic Particle solution (Promega; Binding capacity: greater than 0.75 nmol of biotinylated oligonucleotide (dT) bind per ml of particles) was taken and washed three times with 500 μL saline-sodium citrate (SSC) buffer (0.5×). The washing step was facilitated by applying a magnet to the side of the tube and the supernatant discarded during each wash. SSC buffer with 100 μl of dissolved biotinylated RNA (2 μM) was added to streptavidin-coated magnetic particles and the tube was gently tapped to suspend the beads. The suspended beads were incubated at room temperature for 30 minutes, with occasional agitation by hand. A magnet was applied to the side of the tube and the supernatant was collected. The beads were washed with 100 μL SSC buffer (0.5×) two more times and the supernatant was collected and combined and UV₂₆₀ₙₘ measurement was made (OD₂₆₀=0.123; 300 μL of supernatant wash). Because the supernatant was diluted three times, the OD of the original supernatant must be 0.531. This OD value was compared to the OD of the biotinylated RNA before incubation with streptavidin-coated beads. The yield of the biotnylated RNA was calculated to be 76.8%.

To confirm that the biotinylated RNA was bound to the streptavidin magnetic beads, 0.5 μL of RNAse A/T1 Mix was added to the washed beads in 100 μL of SSC buffer (0.5×). The beads were incubated at 37° C. for 30 mins before the supernatant was collected by applying a magnet. The OD₂₆₀ for the eluted nucleotides was 0.560. The slight increase in absorbance at 260 nm (compare OD of 0.531 for the RNA with an OD of 0.56 for the nucleotides generated from the RNA hydrolysis) is expected as free nucleotides have higher absorption than when in a polynucleotide (hypochromic effect).

### Electrophoretic Mobility Shift Assay

Gel shift assays were performed using 8% acrylamide gels with 100 mM Tris/HEPES, pH=7.5, 10 mM MgCl₂, and 0.1 mM EDTA in the gel and running buffer. Gels were run at 4° C. at 100V for 2 hours. Gels were imaged with a phosphorimager and fraction bound quantified with Fuji Image Guage software. The ³²P cdiGMP probe was synthesized from α-³²P-GTP by incubating overnight with purified diguanylate cyclase WspR (PA3702 from Pseudomonas aeruginosa) in 10 mM Tris, pH=8, 100 mM NaCl, and 5 mM MgCl₂ at 37° C.

It will be apparent from the foregoing examples that we have developed and characterized DRaCALA as a rapid and precise method for qualitatively or quantitatively measuring protein-ligand interactions. We in show in one embodiment the utility of DRaCALA by using the example of cdiGMP binding to Alg44_{PilZ}. The dissociation constant of 1.6 μM obtained by DRaCALA is similar to previous studies using filter binding, isothermal calorimetry and surface plasmon resonance assays. Previous studies of the dissociation rate of cdiGMP from Alg44_{PilZ} were based on saturating the protein with radiolabeled cdiGMP and separating the protein-ligand complex from unbound cdiGMP over a Sephadex column. The half-life of the complex was estimated by filter binding as 5 minutes, which contrast with 35.6±10.7 seconds as detected by DRaCALA. This discrepancy is likely due to two key differences between the two assays. First, DRaCALA is able to directly quantify the total signal in each sample. Because of the various separation steps required for the filter-binding assay, the total ligand in each sample is just assumed to be equivalent. For DRaCALA, the total signal of labeled ligand is known for each individual sample, and therefore eliminates the need to assume that the total signal is equivalent. The ability to detect the total signal and fraction bound significantly increases the precision of the measurement and reduces the error incurred from pipetting and other physical manipulations. Second, the processing times of the assays are dramatically different. The filter assay involved binding, separation of bound ligand from free ligand, filter binding, and the associated wash time, requiring at least five to ten minutes of processing time. DRaCALA directly assays the binding without prior processing or the subsequent wash steps. As a result, DRaCALA can be completed within five to thirty seconds depending on the volume of the sample spotted. Since all binding interactions have off-rates, the speed of the assay is important to capture accurate data. Other techniques for determining biochemical interaction are also available such as isothermal calorimetry or surface plasmon resonance; however, these techniques require dedicated specialized instrumentation and individual processing of samples, resulting in longer assay time and lower throughput. An important feature of DRaCALA is that it will make biochemical approaches accessible to molecular and cellular biologists interested in precise and simple measurements of interactions between protein-ligand pairs of interest. The ability to determine dissociation rate, in addition to dissociation constant, allows calculation of the on-rate. Differences in the dissociation rate can be useful in understanding biological processes since interactions with similar affinities can result in distinct biological outputs.

With respect to the aspect of the invention that entails use of nucleic acids as ligands, it will be apparent to those skilled in the art, given the benefit of the present invention, that nucleic acid-protein DRaCALA utilizes the differential mobility of nucleic acids through nitrocellulose to separate DNA that is bound to a protein from that which is unbound. The interactions we measured in this way were specific to the nucleic acid sequences because point mutations at previously identified critical nucleic acids abolished specific binding of CRP to ICAP in both annealed oligonucleotides and plasmids. The affinity of the interaction was measured by diluting protein with limiting amounts of probe. Remarkably, the K_{d} measured for the annealed oligonucleotide and plasmid closely matched what was reported in a previous study that used a filter-binding assay (Gunasekera, A., et al. (1992) J Biol Chem, 267, 14713-14720.) as well as a study that used gel shift (Fried, M. G. and Crothers, D. M. (1984) E J Mol Biol, 172, 241-262) (Table 9). The off-rate determined with DRaCALA was slower for the plasmid than for the oligonucleotide probe, which is consistent with the finding that nonspecific DNA concentration can affect the kinetics of specific DNA binding with protein. The off-rate for the plasmid (k_{off}=4.84±0.17×10⁻⁴ s⁻¹) was similar to that reported in a gel shift study (k_{off}=1.2×10⁻⁴ s⁻¹). This corresponds to an observed half lives of 23.9 minutes for DRaCALA and about an hour for gel shift. This difference may be explained by the amount of unlabeled competitor used to chase off the probe, which was at 25 times molar excess for the gel shift and 1000 times for DRaCALA. For DRaCALA with plasmid probes, another advantage is that high concentrations of competitor can easily be obtained by PCR amplification. The on-rate cannot be measured using DRaCALA, but it can be approximated with a calculation based on the affinity and off-rate. Using DRaCALA with plasmid probes allows for easy testing of direct binding and specific competition of any potential DNA binding site simply by cloning into a plasmid that can be labeled for detection. Studying kinetics in this system is more analogous to DNA-binding activity in a cell because there is a great excess of DNA to which the protein can bind nonspecifically. One key difference between DRaCALA and filter-binding assay is that for DRaCALA both the bound ligand and the total amount of ligand are measured whereas the traditional filter-binding assay typically only measures the bound ligand. Thus, results of filter-binding assays are typically normalized to 1.0 fraction bound for the highest concentration of protein or ligand. In contrast, results for DRaCALA for the highest concentration of protein is often less than 1.0. There are two potential reasons for the fraction bound detected by DRaCALA to be less than the theoretical 1.0. First, the off-rate of the protein-ligand interaction dictate that during the assay time, the dissociated ligand is mobilized and can not rebind the protein. Second, for all 5′-end labeled nucleotide, a small fraction of labeled free phosphate can be hydrolyzed and appear as free ligand. Because DRaCALA measures both free and bound ligand, the determination for fraction is far more accurate despite the detection of fraction bound of less than 1.0. This does not affect the utility of the method, since the K_{D} and k_{off} that we measured for CRP-ICAP interactions are similar to previously reported results. A similarity of DRaCALA and filter-binding assays is the interaction of proteins with nitrocellulose may alter the behavior of proteins. For DRaCALA, this effect is likely protein specific since soluble and insoluble forms of Alg44 and PelD behave similarly when assayed for binding to cdiGMP by DRaCALA.

Comparing DRaCALA to the traditional separation-based methods reveals some advantages of the new technique. The filter-binding assay was the first popular method that depended on separation of bound and unbound ligands based on differential mobility through a support. This technique was used for the first study of the interaction of CRP with DNA. The electrophoretic mobility shift assay (EMSA or gel shift), which detects interactions because they cause retardation in DNA mobility through a gel, was first introduced as an alternative to the filter-binding assay using the lac repressor as an example. Later it was used to study CRP in greater detail. The major strengths of the gel shift are that both bound and unbound ligand is measured and supershifts provide information about binding structure. A potential issue is the length of time required to run the gel, during which time the protein and DNA can dissociate, which is a particular concern for lower affinity interactions. DRaCALA does not have a wash step and it measures total signal in every sample with a visual readout, making it preferable to the filter-binding assay. EMSA also measures total signal with a visual readout, but requires a much greater assay time than DRaCALA. Although DRaCALA is more rapid, EMSA still retains an advantage in the detection of supershifts that result from an antibody binding to a DNA-bound protein or multiple proteins binding to DNA. The ability of DRaCALA to detect interactions on plasmid DNA is a significant improvement over EMSA, which is most sensitive with probes less than 300 base pairs long.

More modern techniques include chromatin immunoprecipitation on a microarray chip (ChIP-chip) and sequencing of chromatin immunoprecipitated DNA (ChIP-Seq). These assays allow for a high-throughput approach to identify binding sites on the chromosome but provide no measure of affinity and cannot rule out indirect interactions. Because the readout of ChIP-chip is precipitation or a lack thereof, studies of transcription factors such as CRP often have false negatives and include a lot of background noise attributable to low affinity binding sites. The most accurate analytical assays include isothermal titration calorimetry (ITC) and surface plasmon resonance (SPR). ITC uses a controlled chamber to assess heat changes as DNA binds protein, allowing for thermodynamic and kinetic measurements. SPR detects molecular weight changes on a metal surface in real time and can determine affinity and kinetics with remarkable sensitivity. The proof of principle for SPR studies of DNA-protein interaction was first demonstrated using the lac repressor. ITC and SPR have the advantage over DRaCALA in that neither technique requires labeling of the ligand of interest. However, the common drawbacks of ChIP-chip, ITC, and SPR are the relatively high associated costs and need for specialized equipment. DRaCALA uses small amounts of inexpensive materials and requires no special equipment, making biochemistry accessible to molecular biologists. DRaCALA is precise, with standard deviations of measurements that are typically less than 5% of the mean. The value of DRaCALA lies in the simplicity of the technique. The only special tool required is a detector of the label on the probe. Only a small amount of sample and nitrocellulose are needed, making it inexpensive and easy to scale up. Capillary action of small volumes is fast, so separation of bound and unbound ligand takes only seconds. Together, these traits make DRaCALA especially cost and time-efficient in comparison to established methods.

The simplicity of DRaCALA allows adaptation of the technique to study other molecular interactions. PCR conjugation of DNA to a variety of molecules can be achieved using commercially available modified primers, which can have 5′ reactive groups such as aldehydes, amines, and thiols. This can serve the dual function of keeping the molecule mobile through nitrocellulose and providing a mechanism to label the probe in different ways. Radiolabelling small molecules directly is often impractical due to costs associated with chemical synthesis with radiolabeled chemicals, so DNA conjugation could be a good alternative. The free 5′ end of the DNA can be ³²P-labeled as in this study or occupied with a fluorescent dye from a second modified primer in the original PCR reaction. While fluorescence may be desirable for its ease of use, it cannot presently match the sensitivity of ³²P. Bioconjugate PCR was used in this study with the simple streptavidin-biotin system. Biotinylated PCR products were mobile, detectable, and showed specific interactions with CRP and streptavidin. This also allows selective immobilization of biotinylated nucleic acids so that they can take the role of the immobile binding partner in DRaCALA.

As shown in this study, immobilization of RNA allowed detection of RNA interaction with a small ligand. This area has been of great interest since the discovery of riboswitches, cis-acting RNA sequences on mRNAs that directly interact with small molecules and consequently self-regulate their transcriptional termination and/or translation. Such RNAs have been found to bind a variety of small molecules, including amino acid derivatives, coenzyme B₁₂, and the bacterial second messenger cdiGMP. Studies of riboswitches have primarily used in-line probing and equilibrium dialysis to analyze direct RNA binding to its target molecule. These methods require long incubations that limit their accuracy in determining biochemical parameters. Others have used gel shift assays to measure the affinity and kinetics for riboswitches. By comparing DRaCALA to gel shift assays using a Vc2* RNA to establish a proof of principle, we have demonstrated that DRaCALA is a powerful alternative to these methods that is much faster with at least equal accuracy and precision (FIG. 19). In the present example, RNA was immobilized using biotinylation, but RNA could also be immobilized by other means such as with a known binding protein or an additional sequence on the RNA that specifically binds a protein. Another alternative strategy is to use a biotinylated DNA oligo nucleotide that can hybridize with the RNA molecule (3′ end of riboswitch) to provide a method for immobilization. The same technique could also be used to study RNA-RNA interactions in the context of regulatory RNAs, which are ubiquitous in prokaryotes and eukaryotes and have therapeutic potential.

The foregoing description of the specific embodiments is for the purpose of illustration and is not to be construed as restrictive. From the teachings of the present invention, those skilled in the art will recognize that various modifications and changes may be made without departing from the spirit of the invention.

1. A method for determining whether a ligand binds to a protein, wherein the method is performed without a wash step, the method comprising:
a) placing a liquid test composition comprising the protein and a detectably labeled ligand on a dry porous membrane;
b) allowing radial migration of unbound detectably labeled ligand on the membrane; and
c) based on the localization of the detectably labeled ligand on the membrane, determining whether or not the detectably labeled ligand binds to the protein.

2. The method of claim 1, wherein the porous membrane is nitrocellulose.

3. The method of claim 1, wherein the detectable label binds to the protein, and wherein the localization of the detectable label is present in an inner area of a pattern on the membrane, wherein the inner area has greater signal intensity from the detectable label than the signal intensity from the remainder of the total area of the pattern.

4. The method of claim 1, wherein the detectable label does not specifically bind to the protein, and wherein the localization of the detectable label is present in a pattern which lacks an inner area having a greater signal intensity from the detectable label than the signal intensity from the total area of the pattern.

5. The method of claim 1, wherein the test composition comprising the protein comprises a purified protein.

6. The method of claim 1, wherein the test composition comprising the protein comprises a cell lysate.

7. A method for determining whether a test composition comprises a protein, wherein the method is performed without a wash step, the method comprising:
a) placing a liquid test composition comprising a detectably labeled ligand and which may or may not comprise the protein on a dry porous membrane, said detectably labeled ligand having specific affinity for the protein thereby resulting in bound detectably labeled ligand if the protein is present in the test composition;
c) allowing radial migration of unbound detectably labeled ligand on the membrane; and
d) based on the localization of the detectably labeled ligand on the membrane, determining whether or not the protein was present in the test composition.

8. The method of claim 7, wherein the porous membrane is nitrocellulose.

9. The method of claim 7, wherein the composition comprises the protein, and wherein the localization of the detectable label is present in an inner area of a pattern on the membrane, wherein the inner area has greater signal intensity from the detectable label than the signal intensity from the remainder of the total area of the pattern.

10. The method of claim 7, wherein the composition does not comprise the protein, and wherein the localization of the detectable label is present in a pattern which lacks an inner area having a greater signal intensity from the detectable label than the signal intensity from the total area of the pattern.

11. The method of claim 7, wherein the test composition comprises a cell lysate.

12. A method for determining whether a ligand binds to any of a plurality of proteins, wherein the method is performed without a wash step, the method comprising:
a) placing a series of liquid test compositions each comprising a distinct protein and a detectably labeled ligand on separate locations of a dry porous membrane;
b) allowing radial migration of unbound detectably labeled ligand at the separate locations on the membrane; and
c) based on the localization of the detectably labeled ligand at the separate locations on the membrane, determining whether or not the detectably labeled ligand binds to any of the proteins on the separate locations on the membrane.

13. The method of claim 12, wherein determining that the detectably labeled ligand binds to a protein at a location on the membrane comprises determining that localization of the detectable label is present in an inner area of a pattern on the membrane, wherein the inner area has greater signal intensity from the detectable label than the signal intensity from the remainder of the total area of the pattern.

14. A method of separating unbound ligand from bound ligand comprising:
a) spotting a liquid composition comprising a protein and a detectably labeled ligand on a dry porous membrane to obtain a spot comprising the protein, wherein the bound ligand does not migrate from the spot and the unbound ligand radially migrates away from the spot thereby effecting separation of the unbound detectably labeled ligand from the bound detectably labeled ligand.

15. The method of claim 14, wherein the bound detectably labeled ligand is retained in an inner area of a pattern on the membrane and unbound detectably labeled ligand is present in a second area of the pattern.
